(19) United States
(12) Patent Application Publication
(10) Pub. No.: US 2023/0082019 A1
(43) Pub. Date: Mar. 16, 2023

(54) Systems and methods for monitoring brain health status

(71) Applicant: Early Signal, LLC, Cambridge, MA (US)

(72) Inventor: Daniela Brunner, Bronx, NY (US)

(73) Assignee: Early Signal, LLC, Cambridge, MA (US)

(21) Appl. No.: 17/872,719

(22) Filed: Jul. 25, 2022

Related U.S. Application Data
(63) Continuation of application No. 15/442,665, filed on Feb. 25, 2017, now abandoned.
(60) Provisional application No. 62/300,248, filed on Feb. 26, 2016.

(51) Int. Cl. G16H 50/20; G06F 16/25; G06F 16/33; G16H 40/67

(52) U.S. Cl. CPC G16H 50/20; G06F 16/258; G06F 16/3334; G16H 40/67; G16H 70/00

(57) ABSTRACT

Systems and methods for monitoring brain health include determining baseline health of a subject through clinical assessment. A first plurality of observables of the subject across a period of time, comprising a plurality of time intervals, is obtained. Also obtained is a second plurality of observables of the subject across the period. The first and second observables are each one of sensor measurements, measurements of one or more analytes, physiological measurements, and subjective observations. For each time interval, at one least observable in the first plurality of observables and in the second plurality of observables taken during the interval is used to compute a respective brain health index value set for the subject comprising two or more of a motor skill, cognitive skill, mood, and sleep status. A brain health trajectory is determined through comparison of these value sets to the baseline health of the subject at each time interval.

This application claims priority under 35 U.S.C. § 119(e) to application Ser. No. 62/300,248, filed Feb. 26, 2016, the entire contents of which are hereby incorporated by reference.

### FIELD OF INVENTION

The present invention describes systems and methods for analyzing human data related to health and disease and, in particular, a smart self-correcting system that iteratively choses different algorithms and functional domains to provide the optimal answer to at least one of multiple different questions.

### BACKGROUND

Over recent decades, medical research has generated exciting and promising advances in disease diagnosis and treatment. Success of these new therapeutic strategies relies heavily on early diagnosis and treatment, early detection of relapse, or lack of response to treatment and fast adaptive changes in treatment. However, rising costs continue to restrict patient monitoring to intermittent healthcare with diagnostic tests often based on limited patient endpoint measures. Thus, diseases may worsen or change course, or ineffective treatments continued, for extended periods.

The longer disease progression and ineffectual therapy go unnoticed, the more likely that the patient will become refractory to the limited tools that current medicine can offer. Moreover, with the advent of precision medicine, clinical trials are increasingly using patient stratification and adaptive structures. In this setting, discontinuous monitoring limits the speed and efficiency of clinical trials, leads to delays and errors in patient assignments to treatment arms, and extends trial size, duration, and cost. Such limited assessment of outcomes in the clinical setting also leads to bias reporting and high placebo effects, further raising the costs and increasing the risk of failure of development of efficacious treatments.

With the proliferation of smart gadgets an enormous amount of physiological, behavioral and biometric data is being generated on a continuous basis by patients, chief among these the smart cell phone which can be used to measure a broad array of physiological metrics, including body movements and posture, locomotion, and vocalization patterns and language usage. In addition, the consumer market is growing enormously for wearable devices [Ref. 1] that report additional, specific biometric parameters such as heart rate, blood pressure, and blood sugar, with home sensors also being developed [Ref 2].

To date, however, such wearable smart gadgets have been limited to narrow functionalities, such as lifestyle applications (e.g., tracking one's running performance), specific healthcare questions (e.g., adherence to prescriptions or exercise regimens) or tracking discrete readouts for specific diseases that constitute larger markets (e.g., heart rate and Parkinson's disease). That is, a specific problem is addressed with a specific solution, resulting in slow and expensive development of dedicated hardware and software solutions for each healthcare concern.

### SUMMARY OF THE INVENTION

The present relates to the creation of individual health profiles or “avatars” that capture a person's major health domains and that can be used as a surrogate for monitoring health and diagnosing disease, and as a tool to guide decisions and interventions. Such an individual health avatar can be well defined, when many domains are assessed intensively and continuously, or it may become “glitchy” when one or more data streams become sparse, due to, for example, the need to charge or repair a wearable or home sensor. The disclosed analytical system, can ideally still “recognize” a particular health avatar using the information captured from previous data concerning the individual's health variables, their trajectories, and intercorrelations. Missing data thus can be inferred or predicted from past data and thus facilitate analytical work. The present invention relates, in part, to an integrated flexible analytical solution that can capture and therefore define said health avatar, provide fast and accurate answers to questions relating to, for example, evaluations of diagnoses, identification of risk factors, and decisions regarding treatment plans. The disclosed system is ideally a universal smart integrated system that can be tuned to disease signatures at the group and individual level, handle unstructured continuous passively acquired data, be used to answer a myriad different questions, be used in hospitals, clinical trials and in tele-health, be queried to find clinical predictors retrospectively, predict adverse events, be programmed to extract or provide information day-by-day, act as central hub for information processing, and can integrate standard and sensor health care data and “omics” data.

The disclosure provides steps to acquire and format “passive continuous acquisition” wearable sensor data, which is typically “unstructured” and “sparse” data due to different sampling rates and to missing data due to, for example, downtime battery charge needs, technical issues, and varying compliance due to forgetfulness or low acceptability.

The present disclosure relates to a universal platform that can preferably accept data from any smart gadget, for, among other things, monitoring patient health, treatment responses, and improving diagnosis [Ref. 3], and is ideally applicable to a broad range of diseases including, without limitation, neurodegenerative diseases, neuropsychiatric conditions, and cancer. The flexibility of the system allows processing of data and novel queries without major development of specific software. The system provides not only a representation of the health status of a person, but also a health trajectory representing the past and predicting future events, among other things.

In one embodiment, after acquisition of data into an input database, the invention comprises a phase to group experimental data into functional domains (also referred to as domains of function) including, but not limited to motor, cognitive, and physiological functions based on normative data from a control population (constituting “expert domain knowledge”). If domain data, or other data, are not present in a person's dataset, the data not present may be generated based on (e.g., copying) other similar patient data using algorithms to define the missing or incomplete data, and implementing a data imputation step [Ref. 4].

For analysis, a particular query may be chosen, such as:

Is the patient getting better or worse as compared to his or her baseline?

Are the medications and therapies working?

Are there abnormal signs indicating an impending crisis?

Is it necessary for the remote patient to visit the clinic or should a health worker be dispatched to his or her location?

Are participants in a clinical trial showing beneficial or detrimental effects of the experimental treatment?

Should a patient be offered urgent therapeutic intervention based on an alarming deleterious turn in the health parameters?

In one embodiment, functional domains are given appropriate weights per the question being asked. At the same time, multiple analytical algorithms such as, for example, nearest shrunken centroids, support vector machine, penalized logistic regression, random forest, Bayesian Binary Prediction Tree Model and the like [Ref. 5] can be used to analyze the data.

Each algorithm may give differing answers, yet a composite answer may be built by weighting and integrating all answers (e.g., through unsupervised ensemble learning such as averaging, pooling, majority voting, supervised ensemble learning such as stacking, and/or the like [Ref. 6]). In an iterative loop the domains and algorithms may be weighted in different ways until an optimal solution is achieved. In one embodiment, the analysis algorithm may involve a metalearner step that adaptively selects data input and analytical algorithm combinations to improve the answer.

A dedicated and adaptable graphical user interface (“GUI”) allows access at different levels for the person, patient, caregiver, or physician, and for those monitoring ongoing clinical trials. Alternatively, expert users may access the system programmatically, to do manual or automatic queries. An individual, such as caregiver, physician, researcher or the patient may use the answer provided to change a treatment plan (e.g., changing medications and/or their dosages, using or suspending the use of one or more medical devices, performing or canceling the performance of a medical procedure, beginning or suspending therapy, and the like).

The methods provided for monitoring a present or prospective condition of a first subject may comprise:

at a computer system comprising one or more processors and a memory:

obtaining a dataset comprising a first form of physiological or environmental data associated with the first subject in a first format and a second form physiological or environmental data associated with the first subject in a second format;

identifying a plurality of functional domains in said dataset using said dataset;

executing a query to obtain an optimized query answer, wherein said query comprises one or more of.
(i) improving or worsening of the present condition of the first subject,
(ii) a deviation or conformance to a normative group health condition by the first subject, and
(iii) a prediction of an impending positive or negative health event or lack thereof for the first subject, wherein the query is processed by a procedure that comprises:

a) processing the dataset against two or more analytical algorithms in a plurality of analytical algorithms to obtain a plurality of analytical algorithm results;

b) selecting weights for each respective functional domain in the dataset; and

c) applying a metalearner ensemble algorithm to integrate and weight the individual analytical algorithm results to create an integrated answer for the query thereby monitoring a present or prospective condition of the first subject. In some embodiments, the processing a), selecting b), and applying c) may be repeated until the integrated answer satisfies an optimization threshold. In some embodiments, prior to executing the query, the method further comprises structuring any unstructured data in said dataset using a data formatting algorithm. Prior to executing the query, the method may further comprise the step of analyzing the dataset to determine if it is incomplete and, when the dataset is deemed incomplete, the method further comprises imputing additional data points in the dataset, wherein the additional data points are derived from data relating to the subject, a group of subjects similar to the first subject, or a normative dataset. In some embodiments, the method may further comprise comprising treating or modifying a current treatment of the first subject for the present or prospective condition based upon the integrated answer. In some embodiments, the method may comprise treating or modifying a current treatment of the first subject for the present or prospective condition based upon the integrated answer that satisfies the optimization threshold.

The method may further comprise building parts or all of the dataset by, for example, acquiring the first form of physiological or environmental data of the first subject from a first device uniquely associated with the first subject, for a period of time. In some embodiments, the method may further comprise building the dataset, wherein the building the dataset comprises acquiring the first form of physiological or environmental data of the first subject from a sensor uniquely associated with a premise, for a period of time. The period of time may be one minute or greater, five minutes or greater, one hour or greater, one day or greater, or one week or greater. The premise may be a home, a clinic or a hospital. In some embodiments, the method comprises building the dataset, wherein the building the dataset comprises acquiring the first form of physiological or environmental data of the first subject from a sensor uniquely associated with a piece of furniture (e.g. a bed, a sofa, a crib, a couch, a bench, a table, a chair, etc.). In some embodiments, building the dataset comprises acquiring subjective data spontaneously generated by the first subject or generated by the first subject in response to one or more predetermined question posed through a communication device to the first subject. IN some embodiments, building the dataset may comprise acquiring the first form of physiological or environmental data or the second form physiological or environmental data from a location remote to the computer system.

In some embodiments, the present or prospective condition of the first subject is a prospective condition, and the query addresses an assessment of a likelihood of the prospective condition occurring to the first subject. The prospective condition may be a catastrophic health event. The present or prospective condition of the first subject may comprise a disease. In some embodiments, the query addresses a diagnosis of the disease. In some embodiments, the present or prospective condition of the first subject is a trauma that has occurred to the first subject. The query may address an assessment of a recovery from the trauma by the first subject. In some embodiments, the query may refer to a difference in a condition between a first group that includes the first subject and a second group that does not include the first subject.

In some embodiments, the dataset comprises physiological or environmental data of a plurality of subjects. In some embodiments, the plurality of analytical algorithms comprises nearest shrunken centroids, clustering, neural networks, support vector machine, principal component analysis, regression, penalized logistic regression, random forest, and/or Bayesian Binary Prediction Tree Model.

In some embodiments, the first device is a smart phone held by the first subject during all or a portion of the period of time, a smart watch worn by the first subject during all or a portion of the period of time, a wrist band with a wireless transmitter worn by the first subject during all or a portion of the period of time, a physiological sensor attached to the first subject during all or a portion of the period of time, an injectable sensor that is injected into the first subject prior to the period of time, an ingestible sensor that is ingested by the subject prior to the period of time, a shoe sensor worn by the first subject during all or a portion of the period of time, an eye tracking device in visual communication with the eyes of the first subject, a smart-shirt worn by the subject during all or a portion of the period of time, or a computerized textile worn by the subject during all or a portion of the period of time.

The first form of physiological or environmental data of a subject (e.g., data associated with the first subject) may comprise movements of a subject, geographic location of a subject, a cognitive measurement of the subject, a measurement of speech uttered by the subject, a dexterity measurement of the first subject, physiological data of the first subject, a EKG measurement of the subject, an EEG measurement of the subject, or contextual data associated with the subject. In some embodiments, the physiological or environmental data consists of physiological data associated with a subject. In some embodiments, the physiological or environmental data consists of environmental data. In some embodiments, the first form of physiological or environmental may be physiological data and comprises analyte data of a subject obtained through a sensor. In some embodiments, the at least some physiological or environmental data originates in a hospital, a clinic or a home.

The method may be facilitated by a graphic user interface or automated programmatic access.

In some embodiments, the method further comprises the steps of obtaining the dataset from an external data repository. The dataset may comprise data for a plurality of subjects including the first subject and the integrated answer satisfies the optimization threshold when the integrated answer accounts for at least a predetermined amount of variance in the dataset across the plurality of subjects.

In some embodiments, the method may further comprise the step of processing data from said first or second form to determine the presence of missing data and imputing synthetic or replacement data for said missing data.

For example, a patient with a newly diagnosed brain tumor is recruited and accepts to wear a specific device and to run a special Application (“App”) on a smartphone in order to start data collection. Other sensors may be used to allow for passive continuous acquisition of, for example, gait, activity, and sleep experimental data. This data may form a comprehensive profile or health avatar and may be captured by the present invention allowing for a subject's placement on a trajectory diagnostic profile (e.g. a brain tumor trajectory diagnostic profile, a diabetic trajectory profile, a heart disease trajectory diagnostic profile, etc.). For example, based on known data from other patients, and the patient's own baseline profile, it may expected that functional data will be stable over at least the subsequent year. Deviations in the data away or towards the norm (as defined by the trajectory of healthy individuals) are used to monitor progression of disease and potential treatment responses. For example, current imaging methods to track brain tumors are infrequently scheduled and therapeutically inadequate. More frequent analysis of behavioral data is innovative and necessary. Analysis in the platform of incoming streaming data for a patient who has had a brain tumor and undergone treatment may reveal little or no deviation from the baseline health profile. This may indicate an outcome used to reassure the individual about the lack recurrence of the cancer. Conversely, significant deviation from the baseline health profile may indicate the high probability of tumor regrowth. This continuous assessment and feedback to the individual (which may be a closed loop), is not possible in the context of standard health care based on infrequent visits to the doctor's office. Such continuous, frequent assessment greatly improves quality of life as the cancer survivor.

The method may also be used to provide health information or status of patients away from a clinic. A smart device may allow tracking of a patient's gait and respiratory problems, the progression or regression during treatment. For example, patients with Rett disorder that have participated in a clinical trial typically suffer from extreme anxiety and respond negatively to visits to the clinic. Instead of reliance on clinical visits to determine health status, a smart device may track various health parameters without the need of a clinical visit. Additionally, alarms may be sent to the patient or any caregivers (i.e., closing the loop for the care givers), and provides objective data to the clinical researcher (i.e., closing the long loop involving the health care system). Occasionally, one or more of the functional data streams is not captured due to the need, for example, for repair of a sensor. The invention, the analytical platform described here, uses previous data and the remaining sensor data to infer the missing data using the patient's stored health avatar and/or a database of similar profiles. For example, a particular and very subtle pattern of movements may correlate with a life-threatening apnea event, and thus, even if the respiration sensor may not be active, the analytical platform can still trigger an alarm and alert the care givers. In some embodiments, the method may be used to measure various parameters associated with treatment adherence of a patient to allow any member using the system information relating to a patient's adherence to a treatment regimen. In some embodiments, the treatment may be altered based on the adherence of a patient or a cluster. In some embodiments, this treatment is part of a clinical study.

In some embodiments, individuals with a mental disorder such as depression, brain trauma, anxiety, PTSD, Alzheimer's Disease, and other psychiatric or neurodegenerative disorder may purchase or be equipped by their caregivers, doctor, or health system, with a sensor or set of sensors that capture health-relevant data, which can be entered and analyzed using the present invention. The health profile or avatar obtained from such data for the determination of correlations between various signals, the capturing of subtle but reliable patterns or signatures, and prediction of adverse events. For example, a subtle yet consistent signature comprised from sensor readings such as galvanic skin response, cardiovascular, and activity readouts, may be found to be a reliable predictor of a panic attack, a flashback, a nightmare, or a similar such adverse event. The prediction may trigger a number of events, such as a text message to the individual asking if he or she needs help, suggesting a breathing relaxing session, offering a session of a particular therapy know to be effective in such cases, proposing to call a caregiver, or, if the prediction is grave enough it may trigger an alarm sent directly to the caregiver enabling immediate follow up. Such closed loop allows the use of the wearable and home sensors to provide immediate help to the user, enabled by the smart analytical system provided by the present invention.

In some embodiments, it may be the case that an environmental signal explains health signature in a more positive way, e.g., it adds sufficient information such that the event is coded as normal and therefore no alarms, texts, or any such feedback is triggered. For example, the platform may analyze streaming data that suggests a person is experiencing high levels of anxiety, yet the GPS data indicates that the person is in a movie theatre indicating that the response may just be a normal reaction to the storyline. The opposite may be true as well. A signal suggesting high anxiety may be taken as a more serious event if the GPS data shows such person immobile in the middle of a high bridge, where the possibility of a suicide needs to be considered. Other contextual or environmental signals may change the meaning of health signatures. Temperature, for example, is known to affect physiological signals, therefore a health signature that indicates a serious event at 65° F. (such as a raising heart rate may indicate an adverse cardiovascular event), may just indicate a normal reaction to motor activity at 95 degrees Fahrenheit.

Complementing current standard diagnosis techniques. In some embodiments, the system may be used to complement current standard diagnosis techniques. For example, a patient may need to travel a far distance to reach a clinician's office with complaints of a vague nature. Although no diagnosis is offered and frequent follow up and monitoring is impossible or inconvenient, the doctor equips the patient with a smart device capable of various measurements that collects basic or complex physiological and motor function data. A signature in the patient's collected data may be detected through the integrated platform of the present invention in order to allow a medical professional to quickly provide treatment (e.g., urgent remote monitoring and care).

The integrated platform may provide for the development of better and/or more effective therapies. In some embodiments, the integrated platform may allow the correct therapy to be identified for a patient. The ability of the present invention to capture subtle yet reliable health profiles and acute signatures allows for accurate tracking of people's response to treatments and improvement in treatment options. If a clinical trial explores multiple alternative treatments for a disease (e.g., insomnia), data analysis the platform may allow a research to determine distinct clusters of participants in the study which may have more benefit from certain treatments than others. For Example, if an insomnia clinical trial consists of Treatment A comprising exercise, cognitive behavior therapy, and relaxation therapy on a weekly basis and Treatment B comprising the use of a drug such as zolpidem (Ambien), analysis of the data using the present invention allows a researcher to visualize distinct clusters of participants in the study and identify patients of a specific insomnia type which may benefit more from Treatment A than treatment B. These distinct cluster may identify those participants with certain parameters (e.g., physiological and/or biological and/or environmental), for example, low heart rate variability (HRV), high galvanic skin response, and high nocturnal skin temperature tend to have worse nightmare frequencies, which are unaffected by Treatment A, but improved by treatment B. The method may allow researchers to adjust the design of subsequent experiments, and to target a treatment (e.g., a drug treatment regimen) in the clinic to a particular subpopulation that benefits the greatest. The researcher also finds that health signatures are particularly normalized right after cognitive behavior therapy, but unaffected by relaxation sessions. This latter finding helps researchers trim down the behavioral therapy design, and remove the relaxation sessions that add cost but have no beneficial effects.

### BRIEF DESCRIPTION OF THE DRAWINGS

Further features and advantages of the present invention will be apparent upon consideration of the following detailed description of the present invention, taken in conjunction with the following drawings, in which like reference characters refer to like parts, and in which:

FIG. 1 is a block diagram of one embodiment of the invention, which is a system for capturing data, integrating it in a database, and analyzing it as described in the present invention. This particular embodiment depicts a process that utilizes existing and incoming data to optimize descriptive and predictive models, per a given set of queries, and provides optimized algorithms for analysis of streaming data. The platform described in this invention provides, for example, a method for acquisition of data from a unique or a multitude of Data Gathering Devices 1, from External Databases 2, or Additional Inputs 3 (such as but not limited to manual data entered through a Graphic User Interface, or programmatically, from, for example, a clinical laboratory) that connects through a Platform Gateway 34 to a Data Formatting 4 module, and a Context Metadata 5, where it stores subject variables such as name, sex, date of birth, and other information, such as date, time and place of collection and the like. In the next step, Data Formatting 4, it is determined if the dataset has missing values according to the Missing Data Algorithm 6. If data is missing, an Imputation Algorithm 7 may supply the appropriate data using one of two modules, the Feature Domain Knowledge 8 and the Disease Domain Knowledge 9. Once the dataset is complete, it is stored in a Database Complete 10 for future analysis. A Query Module 11 (which can be accessed through a GUI or programmatically) can be used to request a new query, or a query selected from an existing Query Menu 29 (see FIG. 6). The Requested Query 12 triggers the Query Ensemble Module 13 and activates two different modules, a Domain Gain Module 14 and an Algorithm Selection Module 15 that feed appropriate parameters to the Query Ensemble Module 13 to set up appropriate gains for different domains and algorithms. The Domain Gain Module 14 requests and obtains appropriate parameters from the Disease Domain Knowledge 9 module. Once the query is processed, the resulting Query Answers 16 are aggregated through an Ensemble Metalearner Module 17 that provides an integrated answer that may be fed back to the Requested Query 12, though an iterative loop to improve accuracy. Such Ensemble Metalearner Module 17 may request alternative domain gains and/or algorithms to improve the answer accuracy. The final optimal answer is available to the user, report generator, or storage through an Answer Output Module 18. Thus, the Answer Output Module 18 can include not only a GUI but also electronic communication to a doctor office or emergency services. The parameters used for each loop of the training, including the final optimized model parameters are stored in a Trained Algorithms 19 module. Some of the trained algorithms may be amenable to the analysis of incoming streaming data, and are stored in a Streaming Algorithms 20 module. This final module can be accessed online for quick feedback to the user, without the need for algorithm training, or access to the databases, and can also provide new derived data, complementing the original device data, gathered for further processing through the Platform Gateway. It will be understood that any two blocks (e.g., modules, databases, algorithms) connected by an arrow are able to communicate or transfer information via the direction of the arrow.

FIG. 2 is a block diagram of one embodiment of the Data Formatting 4 module shown in FIG. 1. An Unstructured Digital Dataset 21 (shown in the figure as being comprised of 3 different data streams: stream @, stream &, and stream #—where each symbol represents a different data stream that could be, but not limited to, binary or numerical data stream) can be restructured using algorithms to detect and identify events and states to store them in a Semi-structured Dataset 22 where an event can be, without being restricted to, the onset of locomotion, a misstep or a fall, and a state can be, again without being restricted to, walking, sleeping or running. From such Semi-structured Dataset 22 a number of secondary tables can be extracted to further summarize and structure the data. In one embodiment of this invention each data stream can be preprocessed in different ways and stored in a Reformatted Dataset 23. The Reformatted Dataset 23 represents an optional preprocessing step often required to extract derived data from the Unstructured Digital Dataset 21. In an example, the Unstructured Digital Dataset 21 data streams are divided into overlapping windows, or frames, which are denoted with a subscript (_{w}) followed by an index number. For example, stream “@” may contain ECG binary data, whereas stream “@w” may be derived times ECG series data including “@w1” smooth ECG data, “@w2” time stamps for identified peaks (the R peak), and “@w3” could be a series of extracted RR intervals (the interval between two successive R peaks). Another example of preprocessing constitutes breaking the original time series into smaller time series representing a moving window. Basic Statistics Table 24 comprises the first, second and third moments of the variable distributions, such as the number n of events A in data stream @ (n=2), the number n of states I in data stream # (n=3), the mean, variance and skewness of numerical variables, and the like. A Motif Table 25 comprises patterns, sequences, correlations and the like. As an example, a motif may be a set of words in text or speech (such as “you know”, “let me tell you”) or a sequence of movements or events. In some cases, some of these derived measures may be obtained directly from the sensor's APP, or from the sensor vendor cloud service platform. For example, an ECG device may provide a smooth ECG, the time of the R peaks, and the RR intervals, and thus these derived data can enter the system through Platform Gateway 34 rather than being calculated afterwards.

FIG. 3 is a block diagram of one embodiment of the Domain Finder Algorithm 26. Using at least one of the original data gathered through Platform Gateway 34 (FIG. 1), the structured data stored in the Basic Statistics Table 24, and the Motifs Tables 25, a Domain Finder Algorithm 26 is used to find correlations, clusters or other similarly-defined group structures to identify functional domains such as motor function, cognitive function, gait, sleep, etc. Such group relationships may represent the general population (“Norm”) or a subpopulation suffering of a particular disease (e.g., “Disease A”). The domains and associated features are stored in the Feature Domain Knowledge 8 and differences between the norm and various diseases are stored in Disease Domain Knowledge 9.

FIG. 4 shows an Imputation Algorithm 7 in one embodiment of the data formatting steps shown in FIG. 1. The Imputation Algorithm 7 ensures that subsets of data collected at different times from the same subject represent all domains of interest for later analysis. The imputation is done using information stored in the Feature Domain Knowledge 8 and Disease Domain Knowledge 9, appropriately for each disease of for the normative population.

FIG. 5 shows an example of the Domain Sorting Module 28 in an embodiment of the data formatting steps shown in FIG. 1. This step ensures that Domain-heterogeneous Datasets collected at different times for the same subject can be reorganized in Domain-homogeneous Datasets for later analysis and differential weighting by the Domain Gain Module 14.

FIG. 6 shows three example types of queries available in the Query Menu 29. The first query requires extensive personal data for an estimation of a personal baseline. The second query requires extensive population data to assess statistical standing in relation to the population baseline. The third query requires both population and personal baselines to assess personal trajectories.

FIG. 7 is a representation of a Domain Gain Module 14, used to weight different domains consistently with a particular query being addressed and the particular disease between considered. The Domain Gain Module 14 can set the weight given to a domain according to an automated Machine Learning Algorithm 30 or through manual Expert Annotation Module 31 per an aspect of the present invention.

FIG. 8 is a representation of analytical steps comprising the Domain Gain Module 14 that weights the different functional domains and provides such weighted data to the Analytical Algorithms 32. Analytical Answers 33 obtained from Analytical Algorithms 32 are aggregated, and an integrated result is generated by the Ensemble Metalearner Module 17.

FIG. 9 illustrates data calculated from a simulated sleep study involving 200 individuals with one of 3 types of insomnia and a control group. The data is time series data comprising 1000 data points.

FIG. 10 illustrates potential clustering from the data shown in FIG. 9. In these clusters, each node or point represents a cluster of patients. Connections refer to related clusters. This cluster network formed from the data shown in FIG. 9 shows the formation of two large superclusters of points. Each point may have a pattern (e.g., color, size, number, symbol, etc.) to allow visual representation of potential connections between variables to be made. In the cluster network, nodes marked “1” represent clusters of patients with insomnia due to waking up too early, nodes marked “2” represent clusters of patients without insomnia, nodes marked “3” represent clusters of patients who have trouble falling asleep and nodes marked “4” represent clusters of patients who have trouble staying asleep.

FIGS. 11 and 12 and illustrates the same cluster network as shown in FIG. 10 with each node representing another variable for the cluster. FIG. 11 comprises nodes where the size of the nodes represents the number of clusters with more depressed subjects. FIG. 12A demonstrates predominantly male (“M”) clusters and predominantly female (“F”) clusters, which can be seen to be unrelated to the type of insomnia. FIG. 12B demonstrates the mood of each cluster based on the size to help identify alternative hypotheses regarding insomnia type and mood. These and other relationships can be not only explored visually but also statistically quantified to assess their significance. Additionally, these relationships may indicate that a treatment to a patient, a cluster or a supercluster may be improved, changed or altered.

FIG. 13 illustrates a platform ability to separate clusters corresponding to different gestures and that following the removal of possible variability between subjects, more acute and accurate clustering may be obtained.

### DESCRIPTION OF THE INVENTION

### Definitions

As used herein “Additional Inputs” refer to data incoming to the Platform Gateway 34 from sources other than wearable devices or external databases. Additional Inputs 3 may include manually entered data and data contained in laboratory analyses, questionnaires, social media and the like.

As used herein, “acute signature” refers to a health profile obtained using a short to medium time scale used to diagnose, identify, or interpret a subject health status.

As used herein “Algorithm Selection Module” refers to a module that stores or programmatically connects to the stored algorithms to be used in any query. The algorithms connected to may cover all possible analysis needs. Algorithm Selection Module stores information regarding the homology across algorithms, and appropriates weights for use in an ensemble learning context. The weights appropriated by the Algorithm Selection Module to the Query Ensemble Module may be altered by the Ensemble Metalearner as necessary.

As used herein, “analyte data” refers to data pertaining to sensors registering substances, including, for example, biological substances such as glucose, calcium, and the like.

As used herein, “analytical algorithms” refer to process or set of rules followed in calculations or other problem-solving operations to represent the interactions between any variables necessary (e.g., those in consideration), obtain new knowledge and/or derive predictions. Examples include nearest shrunken centroids, support vector machine, penalized logistic regression, random forest, Bayesian Binary Prediction Tree Model and the like.

As used herein, “analytical system” refers to a system that stores and acquires historical, new, and/or streaming data. This system this data to provide reports, visualization, and answers which provide discovery, interpretation, and/or communication of meaningful patterns in the data.

As used herein, “automated programmatic access” refers to data gathering and extraction tools, routines and scripts that can be triggered by an electronic event, such as a schedule or when specified conditions are met.

As used herein, “automatic queries” refer to Queries that can be triggered by an electronic event, such as a schedule or when certain conditions are met.

As used herein, “avatar” or “health avatar” or “health profile” refers to a profile or signature representing a person's health status and characteristics. For example, the health avatar may comprise behavioral, genomics, proteomics, physiological, and cognitive data, and their interrelationships such as their covariance.

As used herein, “Analytical Algorithms” encompasses statistical techniques encompassing predictive modeling, machine learning, and data mining techniques. These may analyze historical, new, and streaming data in order to make predictions, capture patterns, estimate and/or quantify differences in data, quantify time series stability or instability patterns, identify change points in times series, and/or their predictors, and the like.

As used herein, “Analytical Answers” refers to one or more outputs from an algorithm (e.g. Analytical Algorithms) in response to a query.

As used herein, the “Answer Output Module” is optimized output from the Ensemble Metalearner.

As used herein, the “Basic Statistics Table” is a table or matrix or database which stores statistical quantities extracted or calculated from the original data. For example, these statistical quantities may be the moments of the distribution of a variable (such as estimates of the central tendency —arithmetic, geometric, or harmonic mean, median, and mode-, variance, skew, and kurtosis), covariance between two or more variables, etc.

As used herein, “biometric data” is data that can be used to identify a person. Biometric data may include fingerprints, face features, writing or speech characteristics, and the like.

As used herein, a “change point algorithm” is an algorithm designed to detect whether or not a change has occurred, and/or whether several changes might have occurred. The change point algorithm may identify the times of any such changes.

As used herein, a “classifier” is algorithm which assigns data to classes.

As used herein, a “closed loop” is a process by which a user of the analytical system receives feedback (e.g. feedback regarding their health) from some point in the system which changes (e.g. improves) the user's health outcomes. A short closed loop may be exemplified by a wearable sensor, a smartphone that gathers sensors data, processes the sensors data to determine the feedback (using, for example, Streaming Algorithms), and an application on the smartphone which transmits feedback to the user. A long closed loop may involve a doctor, who analyses the platform output before submitting to the user.

As used herein, “confidence” refers to the degree of error expected in analysis. Confidence may be determined by calculating confidence intervals for any output of the analysis.

As used herein, the “consensus result” is the composite answer obtained by weighting more heavily the more frequent and similar answers.

As used herein “contextual data” may refer to data that captures the context in which sensor and other biological or behavioral data were captured such as medication, education of the subject, identity of the subject, genetics of the subject, type of sensor, type of protocol, and the like (see, e.g., Table I). The context may refer to environmental, social, virtual, text, physical, auditory, visual or similar circumstances which define the setting of an event, statement, data or the like, and in terms of which it can be better understood and assessed. The “Context Metadata” module may be stored Contextual data.

As used herein a “continuous transition” refers to a smooth change in the characteristics of an ordered dataset or time series over a short sequence of data input.

As used herein, a “data cluster” refers to a group of variables that have a covariance stronger than that expected from the normative covariance of a whole dataset, unless otherwise specified.

As used herein a “data gathering” device may be, for example, a wearable device, laboratory device, home sensor device, etc. “Data Gathering Devices” refers to one or more data gathering devices.

As used herein, “Data Formatting” refers to modules which provide processes used to adjust, manipulate, complete, or transform the incoming data. The Data Formatting module may aggregate data from disparate sources and prepare this data for insertion into the database.

As used herein “data imputation” may be a process by which incomplete datasets incorporate data to fill gaps or empty records of the empty dataset.

As used herein a “discontinuous transition” refers to an abrupt change in the characteristics of a dataset over a short sequence of data input.

As used herein, “Disease Domain Knowledge” refers to a database containing information about how different functional domains are affected by different diseases, information extracted from historical or new data. This information may be based on external domain expertise, or manually annotated by an expert.

As used herein, “Domain Gain Module” or “Domain Gain Database” refers to a table comprising appropriate optimal weights for different data and queries according to the Feature Domain Knowledge, and Disease Domain Knowledge modules. This Domain Gain Module is utilized by the Query Ensemble Module.

As used herein, “Domain Finder Algorithm” refers to an algorithm trained to find correlations between functional variables that represent different functional axes such as motor, cognitive, cardiovascular, and the like.

As used herein, “Domain Sorting Module” refers to a module or algorithm that integrates different datasets corresponding to the same subject and reorganizes these datasets into predetermined domains.

As used herein, “domains of function” refer to groups of data which reflect a particular underlying process or physiological or functional significance.

As used herein, an “ensemble algorithm” is a machine learning paradigm that uses multiple learning algorithms to solve the same problem. The ensemble algorithm may obtain more accurate and/or quicker results than any of the individual algorithms alone.

“Ensemble Metalearner” refers to a machine learning module that uses and weights multiple algorithms, feature domains, disease domains, and ensemble methods to optimize the answer to a particular query. The Ensemble Metalearner optimizes the answer to specific queries and alters the Algorithm Selection Module and Trained Algorithms as necessary to achieve the optimized answer.

As used herein “environmental data” may be data that captures the environmental circumstances in which one or more sensors and/or other biological or behavioral data were captured. This environmental data may be ambient temperature, humidity, pollution levels, weather, light intensity and the like

As used herein “event” is a change in a physiological, motor, cognitive, health signature or other data that is distinct from variation due to noise or is representative of a longer duration change or state. Thus, whereas “sleeping” is a state, “jump” is an event.

As used herein, “expert annotation” refers to data added to the dataset belonging to a particular subject by an expert human or program, such as type of disease, disease status, diagnosis, and any other such qualifier.

As used herein, “expert domain knowledge” refers to information about a particular area of research, disease, or functional domains representing accumulated knowledge, skill, or authority.

As used herein, “Expert Annotation Module” is a module allowing for manual annotation or assignment of weights based on expert domain knowledge.

As used herein an “external database” may be a database containing data related to health conditions such as health care records, population data, lexicons, demographic data and the like.

As used herein, “Feature Domain Knowledge” refers to stored information regarding the correlation between variables. This knowledge may allow variables to be grouped or weighted, reducing dimensionality, and overfitting.

As used herein, “functional data” refers to data relevant to a functional domain. A functional domain may be the primary division of human functions. These functions may be defined by different organs, their systems and the like (e.g., motor, cognitive, and cardiovascular functions).

As used herein, “glitch” refers to a sudden temporary state characterized by a lower than average level of information.

As used herein, a “health signature” is a set of health variables, their values and interrelations, which characterize and identify a subject health status over a short period of time (corresponding to a slice or snapshot of the Health Avatar).

“Heart rate variability” (HRV) refers to variation in the time interval between heartbeats. HRV may refer to variability of the RR (where RR refers to the interval between the R peak of the QRS complex of the ECG wave) or inter-beat intervals.

As used herein, “homocedacy” refers to the equality of variance for two or more distributions.

“Imputation Algorithm” is a module that imputes synthetic or replacement data to prepare for storage, analysis, or other such process (e.g. for storage in a database).

As used herein an “integrated answer” is a composite answer from multiple sources.

“Kurtosis” refers to the fourth moment of a distribution which is a measure of its flatness. The moment is a quantitative measure of the shape of the distribution. The first moment is the mean, the second central moment is the variance, the third central moment is the variance (or skew), and the fourth central moment (with normalization and shift) is the kurtosis.

As used herein, a “leading indicator” is a measurable variable that changes before the health signature starts to follow a particular pattern or trend.

As used herein, a “learner: is a machine learning algorithm.

As used herein, “longitudinal” refers to a design or protocol in which data is gathered for the same subject or group over a long period of time.

As used herein, “Machine Learning Algorithm” is a module or computer program which learns or extracts non-obvious data from a dataset, such as pattern, predictors, or associations. Machine Learning Algorithm may find combinations of variables that explain phenomena, without being explicitly a program to extract such non-obvious data.

As used herein, “metadata” refers to data about the subject (subject data), environment (environmental data), contextual (context data), and any other detail providing a unique identifier of the dataset of interest (see Table I).

As used herein, a “metalearner algorithm” is an algorithm that uses experience to change certain aspects of a learning algorithm, or the learning method itself to improve the ability to learn.

“Missing data” may be data that was not collected due to inattention, technical difficulty, inconvenience, or any other such possible cause.

As used herein, “Missing Data Algorithm” refers to a module that process data to prepare for storage, analysis, or other such process and finds missing data.

As used herein, a “motif” is a recurrent pattern in a variable or combination of variables, or recurrent subseries in time series, or recurrent sequence of events. “Motifs Table” is a table that stores motifs found in the data.

As used herein a “normative group condition” refers to a state of a group as represented by associated data corresponding to an individual, population, state or event where the data is obtained in the absence of any deviation from normalcy (e.g. in the absence of a disease state, impairment, disorder, etc.). Normative data is data corresponding an individual, population, state or event in absence of any deviation from normalcy (e.g. in the absence of a disease state, impairment, or disorder). Normality refers to belonging to a normally distributed population, or (for a sample) having a distribution that does not significantly deviate from the Normal distribution.

As used herein “omics” refer to any and all fields of study in biology ending with “omics” such genomics, proteomics, and metabolomics.

As used herein, “passive continuous acquisition” refers to the acquisition and/or accumulation of data captured without action from the subject apart from wearing or being close to a sensor, such as heart data, activity, EEG, EKG, EMG, gait, activity, sleep data, galvanic skin response, electrolytes, analytes, acceleration, and the like.

As used herein a “personal baseline” is the state of a subject as represented by associated data corresponding to it most characteristic initial state.

As used herein, “personal data” refers to data belonging to a subject.

As used herein, “Platform Gateway” refers to a module in the platform that collects and/or synchronizes and/or logically joins and/or integrates and/or separates and/or manipulates and/or handles data from one or more sources. The module is a temporary storage for incoming data (cache). The storage may be located in one or more location. Platform Gateways function as a logical gate for incoming data to any modules which separates data to be formatted as necessary and directs the data to the necessary module. For example, metadata may be stored until needed for analysis upon which the metadata passes through a Platform Gateway. This metadata may include adapters from various types of inputs (terminals, internet, Wi-Fi, Bluetooth, etc.) necessary for the Data Formatting input insertion into the database (e.g., metadata necessary for the Missing Data Algorithm.” Platform Gateway functions may comprise requests for fetching data (e.g. from external databases or cloud storage), collection data from any sources, communication with devices to reset/synchronize devices, and collection status identification of inputs (e.g. for starting backup systems or notification to users), can also be used for authentication.

As used herein, the “population baseline” is the state of a group characterized by the same health condition (including lack of disease) as represented by an associated data corresponding to a typical group state.

“Qualification”, “stratification” or “annotation” may refer to the addition of metadata that enables use of subject, contextual or environmental data as part of the analysis or that can be utilized to partition the dataset into smaller, more homogeneous subsets.

As used herein, “Query Answer” is the output from the Query Ensemble Module which may be used by an Ensemble Metalearner.

“Query Ensemble Module” is a module that actively and/or passively processes data with appropriate algorithm weights and selection of appropriate Analytical Algorithms. These weights may be obtained from Domain Gain Module, Algorithm Selection Module, and, directly or indirectly, from Ensemble Metalearner.

“Query Menu” is a set of stored queries for the most common questions posed to the analytical platform.

“Query Module” is a module of the platform that may be used to request a new query, or a query selected from an existing Query Menu representing, but not restricted to, the need to find a change in a subject's health trajectory, diagnosis, prognosis, predictor of an adverse event, differences between groups, effect of a treatment, relationships between variables, or the like.

A “rare” or “neglected” disease is a disease which affects a small percentage of the population. Examples of rare or neglected diseases include orphan diseases. A “rare” or “neglected” question is a question not or sparsely addressed in the literature or for which there is no consensus in the medical or scientific community.

As used herein, “recurrent” refers to the occurrence of an item with probability higher than the average.

As used herein, a “Reformatted Dataset” is a preprocessed data stream that extracts time series characteristics through the rescaling and/or normalization and/or rearrangement of a time series. Reformatted Datasets may extract these characteristics from a smaller subseries, from the calculation of different quantities that are stored and treated as new variables (such as correlation between two or more variables), by moving window calculation results, logarithmic or other such transformations, through change of basis transformations such Fourier or wavelet transforms, compression techniques, dimensionality reduction, and the like.

A “remote” patient is a patient placed at a distance from the clinic or doctor office.

As used herein “Request Query” refers to a module that temporarily stores the selected query specifications, retrieves appropriate weights from Domain Gain Module and Algorithm Selection Module. Request Queries activate and feed appropriate parameters to the Query Ensemble Module.

As used herein, a “Semi-structured Dataset” is a dataset extracted from the original dataset representing extracted obvious or non-obvious quantities such as events and states.

As used herein, a “signature” refers to a combination of related endpoint measures or measured variables and their specific values that represents or identifies a subject, event or state.

As used herein “skew” refers to the third moment of a variable distribution. It is a measure of the distribution asymmetry.

As used herein, “sparse data” refers to data that is infrequent, and/or which presents to any module with highly variable frequency, and/or that presents numerous missing values

As used herein, “stacking” refers to a supervised approach for machine learning ensembles, in which the predictions of various models are trained against the target value, to generate a new combined model.

As used herein a “state” is a change in a physiological, motor, cognitive, health signature or other data that is distinct from variation due to noise or is representative of a discrete activity or event. Thus, whereas “sleeping” is a state, “jump” is an event.

As used herein, “Streaming Algorithm” is a trained algorithm used to process data at the sensor, smartphone, or local computer level. Streaming data is a sequence of digitally encoded coherent signals used to transmit or receive information that is in the process or being transmitted. The Streaming Algorithm may communicate with data gathering devices.

Additionally, alteration of Streaming Algorithms may occur following optimization of Trained Algorithms by the Ensemble Metalearner.

As used herein “structured data” refers to any data amenable to storage in an N-dimensional matrix.

As used herein, “subject data” refers to data that captures the characteristics of a subject such as sex, age, eye color, name and the like. Subjective data refers to data that captures subjective feelings such as happiness, anger, stress, confidence, well-being, and the like.

As used herein, “tabulated data” refers to data stored in an N-dimensional matrix. Structured data may be converted into tabulated data.

As used herein, “telehealth” refers to the acquisition of healthcare remotely via telecommunications technology.

As used herein, a “testing set” refers to a subset of data used to test, as opposed to train, a classifier or model to measure its accuracy.

As used herein, “traditional data” refers to data obtained in a doctor or clinic visits, through phone or personal interviews, or any other such method requiring no sensor.

As used herein a “trajectory diagnostic profile” refers to a profile of a subject which may correlate to a future condition of a patient. For example, a brain tumor trajectory diagnostic profile relates to the probability that a subject may develop or has a brain tumor based on the all are part of the subject's health avatar.

As used herein “Trained Algorithms” are a set of parameters specifying the best result from each round of training, including but not limited to the combination of weights for data domains and algorithms, and specific algorithms parameters.

“Training Sets” are subsets of data used to train, as opposed to test, a classifier or model.

As used herein an “Unstructured Digital Dataset” refers to unprocessed data.

As used herein, “unsupervised ensemble learning” refers to ensemble learning that draws inferences from datasets without labeled responses.

As used herein, “variance” refers to the second moment of a distribution which is a measure of variability, and the average of the squared distances to the mean

As used herein “weighted” data, domains or clusters refer to statistically modified data, domains or clusters, respectively, which are weighted to emphasize or deemphasize its value more than other data.

As used herein, “weighted experts” refer to a combination of trained algorithms or models by way of weighting.

According to the present invention, data gathered in a continuous basis, such as that obtained with wearable device, is used to assess a subject's baseline set of health states and trajectories (where a trajectory is a temporal sequence of states). Wearable devices are well-known and exemplified by smart phones, smart watches, and other such devices [Ref. 7].

Wearable devices, according to the present invention, can be in contact with the subject or carried by the subject (where subject refers here to any human using, intending to use, or potentially using the present invention or similar platforms) on either a continuous basis or with high frequency (where “high” refers to a frequency higher than that used to collect data during visits to a doctor, clinic or the like). The present invention utilizes data from wearable devices, but data may also be obtained from at least one of a smart phone, computer terminal, or other electronic device such as a home sensor [Ref. 8]. It will be understood that complementary data (such as subject data obtained via questionnaires, written or oral, context or environmental data—see TABLE IV and V for data types, can be added at any time to any dataset according to the invention.

### Data Acquisition

Data Input. An input graphic user interface (GUI) may be used to handle collection of the data if such collection needs to be done in a manual or supervised manner. In some embodiments, automatic gathering of data is encompassed by the invention represented by the Additional Input 3 module. Such GUI or input elements may connect electronically to a local or remote Data Formatting 4 module that performs a preliminary analysis to ensure data is in a format compatible with the platforms described by this invention. The Additional Input 3 module may access raw data that may be stored in data tables, and context data, that may be stored in an associated Context Metadata 5.

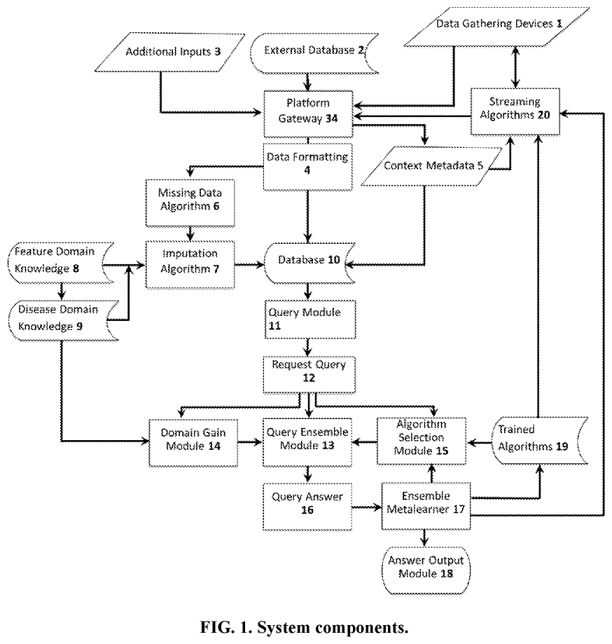

The platform described in this invention provides for acquisition of data from one or more Data Gathering Devices 1, from External Databases 2 (see FIG. 1) in real time (i.e. as the data is being gathered) or post-acquisition (i.e. being transmitted with a delay of varying duration after collection onset), or, additionally, from Streaming Algorithms 20, which can process incoming data to extract features according to pre-existing optimized algorithms. Data can be obtained from existing applications (described herein as “apps”) that can be downloaded through the internet or other electronic networks, from vendor sites (such as the iTunes store), via specialized websites that offer such software, or any other suitable method. Such data can be combined with other data obtained in traditional settings such as doctor or clinic visits, through phone or personal interviews, or any other suitable method. Such traditional data may, in one embodiment of the present invention, be used to complement the smart gadget data and/or to provide contextual data that can be used to qualify, stratify or annotate the data for proper analysis and archival.

Gadgets that are in contact with the subject include, but are not restricted to, smart gadgets, computers, smart watches, electronically equipped bed, crib, wireless headphones, carpet, floor, clothing and the like. Gadgets that are carried by the subject can be attached to the clothing, skin, head, and other body parts, injected, ingested or tattooed. Data can be obtained using sensors built into the gadget (such as, but not restricted to, accelerometers and gyroscopes that are included in many wearable devices), sensors that can be added to the wearable device (such as but not restricted to EKG or cardiac monitor, cortisol and glucose skin sensors), sensors that are independent of wearable devices but provide complementary electronic data (such as, but not restricted to, AutoSense [Ref. 9], a sensor suite that contains sensors to track health activity, breathing, temperature and movement), sensors that can be ingested by the subject to monitor the internal environment, physiological parameters, gut biota, and, but not restricted to, peristaltic movements. Data can be collected by any such sensors, home devices, smartphone-based technology, and signals derived from such raw data are well-known to an expert in the field and are described in the public literature [Ref. 10]. New devices can also be used in conjunction with the platform described herein, as it is intended as a universal and flexible analysis solution.

### Database Formatting

Data Structuring. In one aspect, the invention focuses on the flexibility necessary for the analysis of diverse datasets without undue code or analysis development for a new disease, smart gadget or query. In order to prepare for such generalized analysis, the data need to be presented in a relatively structured format. A key feature of continuous smart gadget data, however, is the production of highly unstructured data. For instance, a subject may produce hundreds of hours of running activity data but not speech data. Another subject may produce several days of EEG data while another may produce none. In one embodiment, the first steps in the process from data input to data analysis result comprise one or more Data Structuring steps.

Data types. ‘Data’ may be any input generated by the subject and or the data input device, whether it is generated spontaneously, or in response to a challenge or query. Thus, examples of data comprise, but are not restricted to, GPS signals, EEG (electroencephalogram), changes of skin electric potential, time of day, and the like. Some data present as Events (where event is exemplified by a fall and comprises data for which duration is of no particular importance), others as States (where a state is exemplified by running and comprise data for which duration is of special interest), and yet others as continuous streams such as EEG. Some data may be analyzed at the level of the electronic device that is also doing the sensing or recording, whereas other data may be analyzed within the confines of the present invention. As an example, consider EKG (electrocardiogram) data: It is possible to perform a basic characterization and analysis at the level of a wearable device that can provide heart rate, an EKG-derived quantity. The EKG and heart rate signals can both be part of the data input. Alternatively, heart rate can be calculated after data is entered into the platform described in the present invention.

Data Stream. A data stream may be any type of data obtained by a particular sensor or a 3rd party data collection platform such as Validic or Human API. Thus, a gyroscope may send a continuous set of numbers through the input step. This Data Stream can be analyzed in an early step to find different Event and States, as defined above.

Raw and Processed Data. Data at the lowest level of processing is the binary data obtained from any data source. Table V shows different levels of processing, including cleaning artifacts (e.g. removing motor artifacts from ECG data), calculating basic quantities (such as counting steps from activity data), or aggregating the data (taking daily averages).

Experimental data. Experimental data may be any data collected that measures or estimates the subjects' Physiological (e.g., EEG), Behavioral (e.g., taping speed), Biometric (e.g., grimace) and other such data. This data may include Objective data, both Continuous (e.g., heart rate, EEG, EKG, gene expression, etc. (see Table IV) and Discrete data (e.g., response to a memory test, taping test, etc.) and Subjective data (e.g., mood, emotion, confidence, etc.).

Metadata. Contextual metadata include, but are not restricted to, the subjects' medication, education, diagnosis, prognosis, time of day, place, disease, and the like (see, e.g., Table IV). Environmental metadata include, but are not restricted to, the ambient temperature and light, humidity, atmospheric pressure, weather, pollution levels, diet, and the like. Subject metadata comprise characteristics that define the subject and are normally unchangeable such as age, sex, race, genetics and the like. Metadata can also include a description of the activities being carried out by the subject prior, during, and planned for after data collection. Metadata can be used, for example, to annotate and properly store experimental data in separated subsets, combined separated data streams into one dataset for each subject, to analyze the data according to different factors, to stratify data and the like. Table IV shows other type of important metadata needed to uniquely identify a dataset.

Primary data comprises the data sent to the system by the Platform Gateway.

Secondary data comprises, for example, any quantity derived from the Primary data, or standardized or processed version of it, such as overlapping sliding windows of a time series, or any other signal for that matter. Thus, for example, if EKG data were the input and heart rate was derived in the system, then they could be Primary and Secondary data, respectively. Secondary data can be calculated with different techniques and may include parameters from model fitting or results from a previous analysis, which can be used as priors. For example, EEG signals or gait time series data may be analyzed using Fourier Analysis or wavelets [Ref. 11] and the resulting estimates can be added to the dataset of a given group of individual. Other features, such as emotion in the case of language processing, or geo-related features in case of GPS analysis could also be extracted. Data can be classified as normal or abnormal, and such classification can also be added as secondary data. Estimates of the moments of the considered variables (mean, variance, skew and kurtosis for instance) and the relationship between the variables (covariance, correlation, mutual information, coskew, and cokurtosis; −[Ref. 12] can also be added as secondary data. In one embodiment, the primary and secondary data form a type of prior set for future analysis. For example, if estimates indicate that a given person shows very stable parameters, (e.g., low heart rate variance), then a new analysis may weigh the finding that heart rate variance is increased more than if such knowledge had not been obtained. The ability to add secondary data adds to the intelligence of an Ensemble Metalearner Module and the system as a whole, as it learns and performs better as more analyses are performed and more primary and secondary data is added.

Data analyzed by the systems algorithms may be referred to as “Features” or “Variables.” For example, a number of features that represent cardiovascular function can be exemplified by heart rate mean, heart rate average, number of arrhythmic events, and the like.

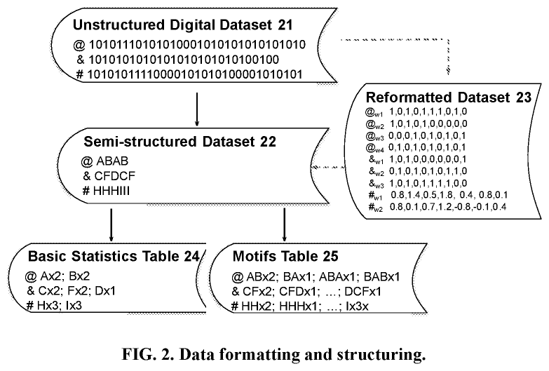

Data Structuring. The invention has the capacity to use unstructured data; it may be necessary to minimally manipulate the data in order to force a structure amenable to data analysis (such has breaking time series data into overlapping windows), although in some embodiments, raw data may be directly subjected to analysis, for example, to look for a particular pattern (e.g., if the question being asked is if the subject ever showed a particular abnormal EKG pattern, the straightforward analysis of the raw EKG may be performed). In many cases, however, there will be a need to combine data from different datasets for the same subject, or to compare against a normative baseline or group and other such analysis that require data formatting. The Data Formatting 4 module (FIGS. 1 and 2) comprises several aspects. An Unstructured Digital Dataset 21 is exemplified as being comprised of 3 different data streams: stream “@” with binary data from the GPS, stream & with binary data from an eye tracking device, and stream “#” with numerical data from EKG—where each symbol represents a different data stream. Algorithms are used to detect and identify events and states as defined above. For example, A=101′ may be identified in DataStream “@” from the GPS, as an event, such as the onset of walking, which may be called event A. In like manner, B=‘011’ is another event in “@.” Events and states are stored in a Semi-structured Dataset 22.

From such Semi-structured Dataset 22 a number of secondary tables can be extracted to further summarize and structure the data. A Basic Statistics Table 24 comprises summarizations (e.g., statistical moments, entropy, and the like) of the feature distributions, such as the number of events A in data stream @ (n=2 in FIG. 2), the number of states I in data stream # (n=3 in FIG. 2), the mean, variance and skewness of numerical variables, and the like. A Motif Table 25 comprises patterns, sequences, correlations and the like. As an example, a motif may be a set of words in text or speech (such as “you know”, “let me tell you”) or a sequence of movements or events. Motifs may be determined a priori, based on experience or the literature or on expert advice, or may be found using pattern-finding algorithms [Ref. 13]. In some cases, a preprocessing step is required, such as data standardization or breaking the stream into overlapping windows (_{wi}) or frames as shown in Reformatted Dataset 23.

### Domain Definition

Functional Domains. One aspect of the invention comprises Functional Domains. A Functional Domain is a set of internal processes and associated behavioral and/or physiological manifestations that allow a subject to satisfy particular internal or environmental demand. For example, cardiovascular function can be considered as a domain represented by heart rate mean, heart rate average, number of arrhythmic events, and the like. As another example, a cognitive domain comprises all central nervous systems process such as neural activity and the like and all associated motor processes necessary to solve a task such as, but not restricted to, learning how to use a computer, learn a new language, or learn how to navigate a new neighborhood. The motor domain, to present another example, includes all internal processes and motor output leading to a particular activity such as locomotion. In some embodiments, features representing different aspects of a functional domain may be associated. For example, a change in the values a feature takes (e.g., heart rate=90 bpm) may be correlated to changes in the values of another feature of the same functional domain (e.g., heart rate variability or blood pressure), although the shape and strength of such correlation may vary widely. The definition of these Functional Domains will be done by reference to an external database or manual annotation or other suitable curating method.

Serendipitous Domains. In one embodiment, features which are statistically associated without belonging to a particular functional domain recognizable a priori may be identified. That is, two or more features may be associated with each other without an apparent reason. This may be caused by lack of recognition of an underlying functional domain, by correlation (or other similarity or dissimilarity measures) between the functional domains that include such features. Such correlation may also be caused by an artifact or systematic bias in data collection or other bias in processing steps, or by association at a very basic physiological and neurological level or the like. In any of those cases, the correlation between features may be an important source of information and, therefore, groups of features, called domains or clusters, will be sought for and characterized. One important feature of content-rich datasets is that they are likely to contain unexpected information, and therefore will maximize the chances that patterns and associations are found in an unsupervised manner. In some embodiments, after analysis, Functional and Serendipitous Domains may be derived from both knowledge-based curating and clustering methods. Clustering methods are algorithms that comb the data to find statistical associations and are known to the expert in the field and exemplified here as correlations, mutual information knowledge, factor analysis, covariance matrices, distance metrics, and the like [Ref 14].

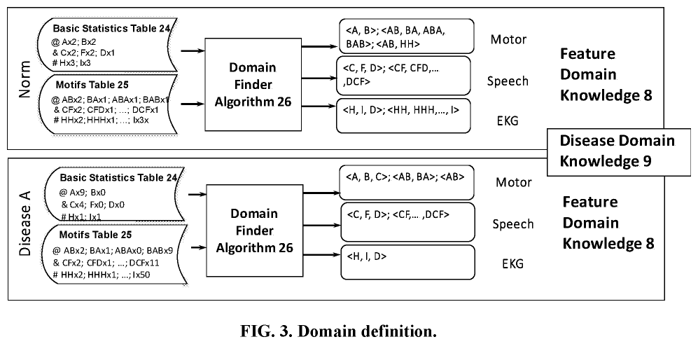

Domain finder. Both Functional and Serendipitous Domains may be found by a Domain Finder Algorithm 26 (FIG. 3) using either the original data gathered through an Additional Input 3 module as in FIG. 1 or the structured data stored in the Basic Statistics Table 24 and Motifs Tables 25. The Feature Domain Knowledge 8 can store all domains in a normative dataset. Domains can be inferred from a normative database (database storing data obtained from subjects not characterized as belonging to a disease subpopulation) or a disease database (data belonging to subjects with a particular disease). For a particular disease, the Domains may have different structure and content and may require different algorithms for extraction of pertinent information. The relationship between features and domains is stored in the Feature Domain Knowledge 8 table. The relationships between diseases and their associate Domains are stored in a Disease Domain Knowledge 9 (FIG. 3). Disease Domain Knowledge captures specific Feature Domain Knowledge 8 tables for each specific disease. As an example, walking pace and body temperature may be unrelated in a normal subject, but highly positively correlated, or inversely correlated in a subject having a particular disease. Both the Feature Domain Knowledge 8 and Disease Domain Knowledge 9 can be curated by an expert in the field (e.g., a key opinion leader, a healthcare professional, a social worker, an epidemiologist, etc.) to provide external knowledge, to verify the found relationships, or to interpret them.

Intra and Inter Domains. Domains may thus be represented by groups of features that are correlated in a measurable quantity. Information regarding the correlation between such Domains is also of importance (for example, the association between general arousal and motor coordination) and is captured and stored in the Feature Domain Knowledge. Association between Domains is by definition weaker than feature associations within Domains. Optimally, Domains are defined, in one embodiment, such that the total variance in the dataset is maximally explained (i.e., accounted for) and partitioned into intra and inter Domain variance.

### Imputation

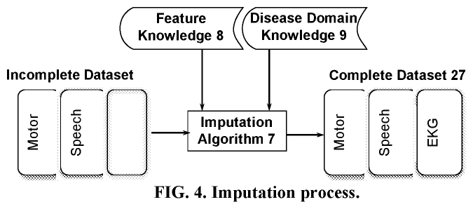

Missing data. In a Data Formatting 4 step, it may be determined if the dataset is complete or has missing values according to an analysis performed by a Missing Data Algorithm 6 (FIGS. 1 and 4) that combs the data and returns a flag for each data cell that remains empty after data entry. If data is missing, an Imputation Algorithm 7 (FIG. 4) can supply the appropriate data using Feature Domain Knowledge 8 and or Disease Domain Knowledge 9 as appropriate, or other suitable algorithms such as replacement by the group average, by a predictive model trained using available data against the variable to impute, or the like, in different embodiments of the present invention. The availability of Feature Domain Knowledge 8 may imply having previous information about association, correlations, and other type of informational relationship between features (captured in the Domains) in order to that allow an algorithm to obtain the most probable estimated value for the missing data. Such estimate may originate from a subject's own data, from a subpopulation of subjects having a similar health status, or from a normative dataset. The Imputation Algorithm 7 in an embodiment of ensures that subsets of data collected at different times represent all domains of interest and provides a Complete Dataset 27 for later analysis.

### Domain Sorting

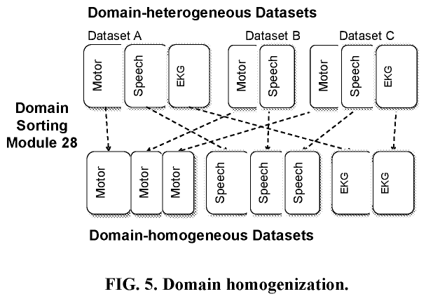

Before analysis, a final step in the organization of data can include a Domain Sorting Module 28 (FIG. 5). This step ensures that subsets of data collected at different times can be reorganized [Ref. 15] in Domain-homogeneous Datasets for later analysis and differential weighting by the Domain Gain Module 14.

### Data Analysis

Query. Once a Complete Dataset 27 is obtained, it may be stored in a Database Complete 10 for future analysis. A Query Module 11 can be used to request a query through a GUI, for example by having a user select from an available Query Menu 29. Alternatively, queries can be made by programmatic access to the system. The Requested Query 12 triggers the Query Ensemble Module 13 and activates two different modules, a Domain Gain Module 14 and an Algorithm Selection Module 15 that feed appropriate parameters to the Query Ensemble Module 13.

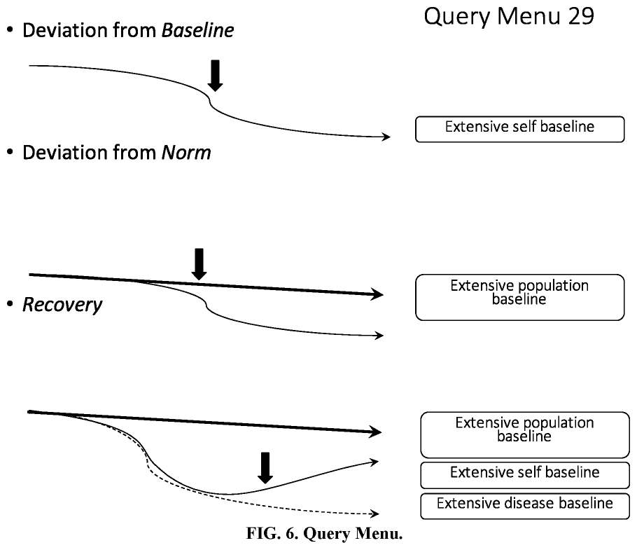

FIG. 6 shows an example of three types of queries available in the Query Menu 29. The first example query “Deviation From Baseline” interrogates the system about the current state of an individual in reference to her historic health trajectory, and requires extensive personal data for an estimation of a personal baseline. The second example query “Deviation From Norm” expects an assessment of the statistical standing of an individual in relation to the population baseline, and requires extensive population data. The third example query “Recovery” assesses a personal trajectory against both the normal population and a disease subpopulation baseline to determine if a particular subject shows the beneficial effects of treatment. Each requested query therefore accesses an appropriate dataset or a slice of one dataset. Datasets can be set automatically or manually by an expert in the system. For example, analysis of the health trajectory of an individual may be required for the duration of a 2-month study, but an expert may inquire about the results using simply the last week of recording.

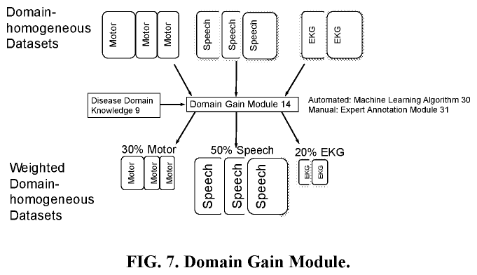

Domain Gain Assignment. The Domain Gain Module 14 may request and obtains appropriate gains or weights from the Disease Domain Knowledge 9. For example, if the disease of interest is a motor disease, the Disease Domain Knowledge 9 will feed a high gain for motor domains and lower gains for other domains. The Domain Gain Module 14 can then weigh the data appropriately (FIG. 7). Thus, motor data will be given a high weight and data belonging to another cluster or domain will be given lower weights. Consistently, associated Domains are given similar weights. In some embodiments, the Domain Gain Module 14 can set the weights following exactly the relationships found in the Feature Domain Knowledge 8 and/or Disease Domain Knowledge 9 tables adjust them according to different automated Machine Learning Algorithm 30 or through manual Expert Annotation Module 31. As an example, a consensus may be found in the literature that for a disease the motor domain is the most important, yet the data may suggest that better results are obtained when the cognitive domain is given a higher weight. The system therefore can start an analysis using stored weights but modify them as needed.

Algorithm weighting. The Algorithm Selection Module 15 activates different algorithms for analysis and, importantly, can give higher weights to particular algorithms according to the Requested Query 12 and to the disease of interest. For example, a multiple regression analysis or other method may be used to extrapolate and predict where the subject would be at a particular time in the future and such prediction can then be compared with the actual data collected at the target time. If the comparison yields a significant difference (where significant means that the deviation from the predicted value is larger than a deviation expected simply due to chance) then the subject's health is deemed to be worsening or improving, depending on the query selected and the dataset being analyzed. Such multiple regression analysis may be optimal for certain diseases but not others. A variety of appropriate Analytical Algorithms 32 may be used for each query. The specific Analytical Algorithms that are used can be set programmatically by the Algorithm Selection Module 15, according the specifications of the Requested Query 12, or set manually in a different embodiment of the present invention.

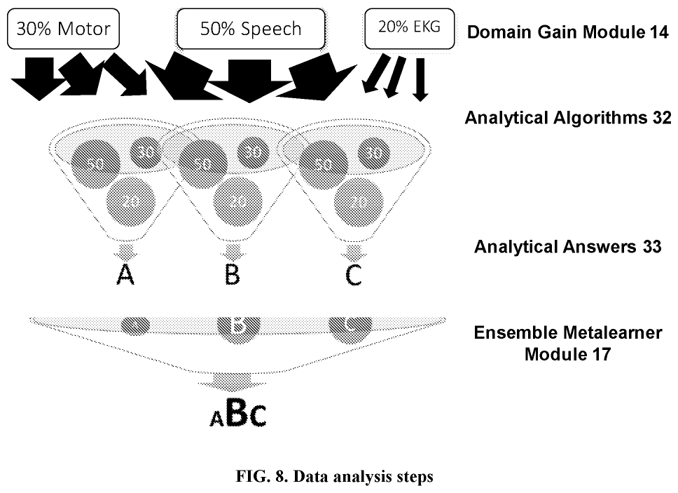

Result integration. The Algorithm Selection Module 15 not only can activate different Analytical Algorithms 29 but it can also weigh the Analytical Answers 33 and integrate the results (FIG. 8). The integration of the results produced by the different analysis algorithms can take different forms such as boosting, bootstrap aggregating (bagging), ensemble averaging, stacking, etc. In one embodiment, the results are simply weighed and averaged by the Ensemble Metalearner Module 17 and the resulting sum is presented to the user through the Answer Output Module 18. For example, algorithm A gives a result R_{A}=80% (meaning that the chances of having recovered from an illness are 80%), and algorithm B, R_{B}=40%. Algorithm A may be preferred for the subject's particular disease and algorithm B may have been found to be somehow useful in previous studies. Thus, algorithm A is given a weight w_{A}=0.8 and B is given w_{B}=0.2. The final integrated result is:

R_{A,B}=R_{A}×w_{A}+R_{B}×w_{B}=0.8×80%+0.2×40%=76%, Equation 1

where w_{A}+w_{B}=1. In another implementation, a majority vote can be implemented. In a different example, if algorithms A, C and D predict that the subject is improving, and algorithm B predicts no change, a majority vote states that the subject is improving, consistently with 3 out of 4 predictions.

Optimization by an Ensemble Metalearner Module. Once the query is processed the resulting answer is improved through an iterative process triggered by Ensemble Metalearner Module 17. Such algorithm may request alternative domain gains and or algorithms to improve the answer accuracy. The final optimal answer is available to the user through an Answer Output Module 18. Optimizing the answers in a dynamic way is one embodiment of this analytical platform. Various techniques can be used, of which a few are described here by way of example:

In one embodiment, optimization can be performed in a supervised manner, when the truths are known (such as in a retrospective analysis, or by using newly imputed contextual metadata or the like). In other words, some of the analyses benefit from availability of metadata confirming membership to a particular class such as disease versus health class. That is, some subjects are already known to belong to a disease class and thus their signatures can be used to train a classifier to recognize such disease profile. A new subject with an unknown diagnosis may present with abnormal data, prompting the analytical platform to classify his data as belonging to a particular disease class. Once the subject is seen by his doctor and further analyses confirm the analytic platform diagnosis, such confirmation can be added as new metadata to the system. The combination of domain weights and algorithm weights used (which is always stored for each query in the Trained Algorithms 19 module) to produce successful classifications or diagnosis can then be preferred for further analysis for similar queries. In this way, the more the system is used and its results are contrasted with new data, the more this learning process improves classification and prediction accuracy.

Further optimization is possible when new algorithms are added to the system and old queries are reanalyzed. In one embodiment, the optimization process is performed on a frequent basis to ensure the data is always analyzed in the best possible way. Users can be automatically notified if a new analysis finds new patterns of importance, previously unnoticed.

Optimization can be done in a supervised manner, when the truths are known (such as in retrospective analysis, or by using newly imputed contextual metadata or the like). The system can be optimized in an unsupervised by improving the model's fit to the data (such as a subject's trajectory) or increasing the variance explained. For example, a subject's trajectory may be fitted using regression methods and the final model accounts for 60% of the variability in the data. As this is considered a poor fit (according, for example, to fit criteria stored in the system) the Ensemble Metalearner Module 17 may conduct parameter search and may trigger a new analysis loop using different weights for domains (e.g., weighting more the motor function data), new algorithms weights (e.g., weighting more change point algorithms), and/or new ways to combine the algorithm answers (e.g., changing from a simple majority voting of results to a weighted average), until it converges to a higher level of explained variance.

The manner in which algorithms are combined can be dynamically improved by analysis of the correlation between their answers. Combining answers from multiple non-independent algorithms may produce a suboptimal solution to a query. In some embodiments, it is preferable to have fewer independent algorithms that many correlated algorithms. The ability to explore correlations between algorithms in a large dataset allows the examination of their interdependence. For example, simple and polynomial algorithms could be reasonably expected to be non-independent. Indeed, both provide for a linear estimate of a trajectory, as shown by equations 2 and 3:

f(x)=ax+b Equation 2

g(x)=cx²+dx+e Equation 3

The terms ax and dx will necessarily provide for a degree of co-variance between the two regression functions.

If such linear estimate is strong and wrong, combining the two algorithms using a simple average will produce a very linear, and thus very wrong, answer. This is especially true if there is a better alternative algorithm, such as one based on mutual information, in which linearity is not necessarily present. Not weighing the three answers will give the best non-linear algorithm only ⅓^{rd} of the contribution, and the rest ⅔^{rd} to the answers with strong and wrong linear estimates.

Weighting the answers for such covariance using the estimated correlation (or similarly derived coefficient), can help solve the problem and reduce the amount of error produced by dependent algorithms contributing to a combined solution.

For multiclass algorithms, there could be lack of independency for a set of classes but complete independence for a different set of classes. For example, algorithm A and B may provide the exact same classification of data into classes 1, 2, and 3. For example, it could happen that subjects number 1 to 10 are classified into class 1 corresponding to “healthy” subjects, subjects 11 to 20 into class 2 for “Alzheimer's Disease”, and subjects 21 to 30 into class 3 for “Huntington's Disease” by both algorithms A and B, in a possible multi-group classification query. Yet, the two algorithms give very different results for classes 4 and 5. For example, algorithm A may classify a random set of subjects n into class 4 corresponding to “Parkinson's Disease” and the remaining into class 5 or “Frontotemporal Dementia” class, whereas algorithm B could classify an independent and different random set of m subjects into class 4 and the remaining into class 5. In this case then, algorithms A and B answers are in a way redundant for classes 1, 2 and 3 (with correlation r²=1), but informative and different for classes 4 and 5.

As an example, consider the above case with the addition of algorithm C, which is completely independent from both algorithms A and B for all classes. When classifying a novel sample, from a subject not used to train the algorithms, let's assume that algorithm A, B and C give the next set of scores for each class:

TABLE I
Algorithms A, B, and C scores for each class

| Scores | Class 1 | Class 2 | Class 3 | Class 4 | Class 5 |
|---|---|---|---|---|---|
| Classifier A | 0.26 | 0.15 | 0.10 | 0.25 | 0.24 |
| Classifier B | 0.26 | 0.15 | 0.10 | 0.20 | 0.29 |
| Classifier C | 0.26 | 0.40 | 0.04 | 0.30 | 0.00 |
| Average | 0.26 | 0.23 | 0.08 | 0.25 | 0.18 |
| R(i)_{A, B, C} | | | | | |

A simple averaging (shown in the bottom row of Table I) gives the combined scores for each class I using the three algorithms, R(i)_{A,B,C}. A simple majority vote will determine that the novel sample belongs to Class 1, as R (l)_{A,B,C}=0.26>R(j)_{A,B,C} for j=2, 3, 4, and 5.

To account for the correlation between algorithms A and B for three of the five classes, we construct weights (Table II) and apply them before combining.

TABLE II
Weights for algorithms A, B, and C for each class

| | Weighted Probabilities | | | | |
|---|---|---|---|---|---|
| | Class 1 | Class 2 | Class 3 | Class 4 | Class 5 |
| Classifier A | 0.25 | 0.25 | 0.25 | 0.33 | 0.33 |
| Classifier B | 0.25 | 0.25 | 0.25 | 0.33 | 0.33 |
| Classifier C | 0.50 | 0.50 | 0.50 | 0.33 | 0.33 |

The resulting weighted combination Rw(i)_{A,B,C} (weighted expert”) is shown in Table III

TABLE III
Algorithms A, B, and C weighted scores
for each class and weighted combination

| | Weighted Probabilities | | | | |
|---|---|---|---|---|---|
| | Class 1 | Class 2 | Class 3 | Class 4 | Class 5 |
| Classifier A | 0.07 | 0.04 | 0.03 | 0.08 | 0.08 |
| Classifier B | 0.07 | 0.04 | 0.03 | 0.08 | 0.08 |
| Classifier C | 0.13 | 0.20 | 0.02 | 0.10 | 0.00 |
| Rw(i)_{A, B, C} | 0.26 | 0.28 | 0.07 | 0.25 | 0.18 |

A simple majority vote now determines that the novel sample belongs to Class 2, as R(²)_{A,B,C}=0.28>R(j)_{A,B,C} for j=1, 3, 4, and 5. Note that removing the influence of the correlation between algorithm A and B for the first three classes actually changed the prediction in this example. For N algorithms, an N×N table of correlation coefficients for each class can be build and used as basis for the weighting. The scores from Table III can be normalized and interpreted as probabilities, although such extension is not needed for simple majority vote or other ranking combination methods.

For non-independent algorithms, different types of training sets (where training sets are subsets of the data used to train classifiers, as opposed to testing sets which are subsets of data kept aside to assess the accuracy of trained classifiers) may be used for each classifier in need of training, to reduce the amount of correlation between trained algorithms and reduce classification error due to inter algorithm-dependencies. In another embodiment, this can be accomplished through the training sets using only a subset of the available features from each domain to train the different algorithms, thus providing again some variability in the ability of the trained classifiers to model that data, and make predictions and classifications. Features can be withheld uniformly across domains (feature reduction) or from a particular domain (domain reduction). Diversity between training sets can also be achieved by resampling the original dataset with replacement (bagging), thus artificially and differentially enlarging the different training sets.

Confidence and Statistical Significance. Machine learning algorithms are notorious for their tendency to over fit data if not carefully used. Over fitting results in seemingly meaningful patterns in the data that are not confirmed or replicated when a different independent dataset is analyzed using the same trained algorithm or model. Discarding real differences between the datasets, this may just mean that the algorithm found a pattern in the noise of the data, that is, in the data fluctuations that have no relation to the experimental situation or question under study. Other modelling techniques may also provide answers to experimental queries that may be wrong or misleading. A way to judge the results of an analysis is to calculate what will be expected under a different scenario. For example, if a researcher is investigating differences between two groups, an important alternative hypothesis is the Null Hypothesis (symbolized with Hₒ) that states that the two groups do not differ from each other. Under Hₒ (i.e., if Hₒ were true) it is possible to obtain a distribution of possible algorithm or model answers that are simply due to chance. The ability to predict what would be obtained under Hₒ allows a comparison between the result obtained and what could be obtained by chance, and can be used to build a confidence index, such as a p-value (which represents the probability that a result is due to chance, assuming that the assumptions of the model or algorithm, such as homocedacy or normality, were met). It is also possible to using bootstrapping to produce predictions for many subsamples to build a confidence interval for the model predictions. In a classic permutation test, the distribution of such model predictions can also be compared with similar predictions obtained with a randomized labels dataset (in which the values of the informative variable are assigned to the subjects randomly). The overlap between the distribution of the predictions using the original labeled subsets and the distribution obtained with the randomized labels subsets gives and index of confidence in the results (with little overlap indicating a small likelihood that the original results are due to chance). The value of permutation tests is that there is no need to make assumptions about the data (normality for instance) and no need to resort to theoretical distributions (such as F, t, or Chi Square) that have a strong dependence with underlying assumptions. Permutation techniques and the like are therefore amenable to many different techniques and are not restricted by data or model assumptions. Also, in general, an index of confidence is the proportion of variance in the dataset that is explained by a model (such as omega square for regression models). One of more of these techniques can be used to estimate confidence which can then be part of the output of the platform. Other indexes of confidence can be built, as well. Another way to assess results, for binary classifications, is to calculate the positive and the negative predictive value (PPV and NPV, respectively; or percent of true positive or negative classifications over all positive or negative classifications, respectively), and their ratio. These indexes can be used to incorporate the notion of prevalence and Bayesian statistics, into measures of confidence. Confidence indexes can then be used in a loop to improve the predictions by an operator, or programmatically by an Ensemble Metalearner 17 algorithm. Confidence indexes can also be used for the decisions to trigger alarms or feedback to the users (e.g. a result with a confidence index below a given threshold does not trigger an alarm).

Why is the Analytical System Particularly Smart? In most embodiments, the invention results in high accuracy of health tracking, diagnosis and prognosis due to its various levels of adaptive designs: first, appropriated handling and integration of continuous and discrete data; second, a set of intelligent machine learning and standard algorithms to provide a fit to differing aspects of the data; third, the ability to focus on the most important features for each disease and type of query; fourth, an integrator step converting individual answers to ensemble results; and fifth, a metaloop ensuring that all parameters can be improved and that the system can learn from its owns failures.

In another embodiment of the present invention, the system can be used to diagnose new diseases by comparing individual health trajectory against the varied disease group trajectories and/or characteristics stored in the system's knowledge tables.

In another embodiment of the present invention, the system can be used to provide on line or delayed feedback to the subject regarding his or her health status, alarming conditions, expected beneficial or adverse events and other such predictions.

In another embodiment of the present invention, the system can be used to monitor infants collecting data through wearable devices in contact with or without their knowledge to their body and/or clothing.

In another embodiment of the present invention, the system can be used to monitor a bed or crib equipped with sensors. Such embodiment would be preferred to monitor infants diagnosed with a particularly dangerous condition such as, but not restricted to, Rett disorder (to detect apnea episodes, for example) and Tuberous Sclerosis Complex (to detect infantile spasms and/or seizures, for examples) or recovering from a medical procedure, or for simple monitoring of a normal infant function.

Individualized cognitive function monitoring is central to medical sciences, as cognitive function is often one of the first domains to be affected. For example, in Huntington Disease (HD), cognitive function shows deterioration up to 15 years prior to diagnosis [Ref 16]. Technologies, such as cognitive applications in smart devices, have focused on discrete sessions to perform assessment of cognition to diagnose or track cognitive function in a number of disorders, patients thus being monitored only in an irregular and discontinuous fashion. Although some tests have been developed to assess these functions in the lab with standardized experimental protocols, no continuous monitoring version exists, in particular, one that takes the advantage of wearable technology. This invention also provides a method for the detection of early signs of cognitive dysfunction amenable but not restricted to a health-monitoring solution using cell phones or other wearable smart device. Assessment of cognitive function is, however, particularly tricky, not easily applicable to noninvasive, continuous gathering of data in the cognitive domain. Visual Function: Despite that visual spatial impairment is often an early symptom of neurodegenerative disease, such as HD, Alzheimer's disease, Parkinson's disease, Lewy Body Dementias, Corticobasal Syndrome, Progressive Supranuclear Palsy, and Frontotemporal Lobar Degeneration, this domain it is not well-assessed by current tests nor it is used for diagnosis, monitoring or treatment evaluation. Neurons in the central nervous system respond to orientation, spatial frequency, color, geometry and other aspects of objects in the visual field, and thus degeneration in the visual association areas and associated circuits affect the way visual stimuli creates our rich visual experience and thus affect behavior, creating a cascade of deficits including inappropriate shifts of attention, lack of inhibition of irrelevant information, lack of gathering of important visual, and or inappropriate sensory-gating of environmental stimuli [Ref. 17]. Thus, if the visual system does not trigger automated tracking and gathering of information through attentional systems, a subject may not be able to successfully plan a motor trajectory through the environment that successfully navigates among obstacles. The present invention takes advantage of the robustness and simplicity of assessment of such basic processes, e.g. visual scanning and sequencing that can be done while the subject is engaged in normal, daily life actions, in both a discrete or continuous assessment fashion. Of particular interest is eye gazing in different environment, which can capture exploration of noel environments and search for needed objects in habitual environments. Eye gaze can be tracked using special glasses or small wearable cameras, or monitored via cameras external to the subject, and the novelty of the environment can be assessed using the GPS signal and a record of explored and unexplored locations. Tracking of eye gaze can be improved by also tracking the relative position of the eyes to the body center. Self-centered and Landmark Maps: Subject transverse the environment and locate themselves relative to other environmental elements. Environmental landmarks, in turn, are encoded in relation to each other, forming a relative reference or cognitive map. The self-centered map, and relational landmark map are updated as the subject moves through the environment, and become consolidated in memory as trajectories become routine, ceasing to utilize attentional processes. Eye, body, or movement trajectories therefore change as the environment and trajectories through it become habitual. These two reference frames depend on different brain areas and circuits and thus deficits in one or the other could be used for precision diagnosis. Of particular interest is the change in the convolutedness of the trajectory as it goes from being novel (likely to be complex, jerky, convoluted) to being habitual (optimal, simpler, and perhaps straighter). This can be captured using the GSP and a record of explored and unexplored locations. Language: Language is a crucial component of our intellect and reflects education, memory and cognitive function. Minor damage to the CNS can result in abnormalities in intonation, tone, stress, rhythm, conveyed emotions, the forms used (such as statements, questions, or commands), the use of irony or sarcasm, emphasis, grammar, choice of vocabulary, or other aspects. Capturing how speakers actually speak and or write, or simply choose words and their sequence, can reveal underlying pathological processes representing onset, progression or even recovery from disease [Ref. 18]. Elements that can be used to assess cognitive function are the frequency of words, phrases, collocates (words that appear close to each other), variation of language and n-grams (i.e., sequences of words that are associated in normal language) and other aspects of language. The present invention can incorporate aspects of speech, writing, language use and language-related memory, and word and concept associations. Language, written and spoken can be captured by monitoring conversations in a smart phone, interaction with AI virtual assistants (such as Amazon echo and google home) or through other wearable devices. The GPS can also be used to qualify the environment as novel or habitual, or even to note if the signal is being recorded at home, park, clinic, movie theatre, or other place, allowing such integrated information to be used as metadata for analysis, as a change in environment is likely to affect the way subjects expressed themselves.

In another embodiment of the present invention, the system can be used to monitor signals originating from wearable devices specifically designed for the system such as special shoes to measure subtle changes in gait or motor movement and coordination in Rett disorder, other disorders in which gait or motor function is affected, or in normal subjects. Such device will, for example, comprise two or four sensors, one on each shoe or limb that will provide signals indicating the relative position and movement of the feet or limbs such that aspect of gait can be extracted. For example, the typical “hand flapping” (quick flapping motions of the hands, usually bending from the wrist) of girls with Rett syndrome could be captured triangulating two hand-positioned sensors with a third sensor placed in the body, to continuously estimate relative position of the hands and their movement. A third sensor providing a GPS signal can complement the limb signals to give a complete motor trajectory. The GPS can also be used to qualify the environment as before. This is important as healthy individuals, those with neurodegenerative or developmental disorders and the like will change body movement behavior in response to different environmental or social situations. For example, an increase in hand flapping may indicate heighten stress, or an unsteady gait may indicate a response to a novel environment for those with a neurodegenerative disease. Tracking Sequences. An example of a method to capture cognitive function is to use the eye gaze or other responses to follow attention to elements of a sequence, such as words or objects presented on a screen, iPad, smart phone or other such device. If such objects are words, based on the common n-grams (i.e., sequences consisting of an integral number (“n”) of words), it is possible to track if people are using acquired language or if their choice deviate from the expected. Thus, for example, after the word “the” is presented at the beginning of a sequence, it will be expected that the word “boy” is chosen instead of the word “before”, if such pair is presented right after the word “the”. In this way, either a click on a touchscreen or pad, or attention as measured through eye gaze, to such objects can be used to follow n-gram (sequences of words that are associated in normal language) choice “trajectories.”

One embodiment of this invention combines data from different input devices to create signatures specific to various environmental conditions. For example, it is of particular interest to distinguish signatures of body or limb movements, series of choices, trajectory of eye gaze, and the like in novel versus familiar environments, or relaxed versus stressful conditions.

Visualization. To add in the investigation, identification, definition, and quantification of health signatures it is important for the user, researcher, and caregiver to be able to visualize the data and the results of the data analysis. Various forms of visualization can be used as part of the platform including scatterplots, bar charts, pie charts and the like. Of interest are charts depicting trends over time such as daily measures of heart rate and heart rate variability. However, what are more difficult to depict are the correlations between variables, and the changes in the associated correlation matrix, particularly in high dimensional datasets. For example, heart rate and heart rate variability may vary significantly from day to day for a given subject according to levels of activity. Such relationships may be crucial for the determination of disease status and trajectory, and thus it is an important aspect of the platform to provide a visualization of the interdependencies of multiple variables (in this example, three variables: heart rate, heart rate variability, and activity). The resulting multidimensional space can be depicted as a point cloud, in which each point represents a patient with coordinates corresponding to the various readings.

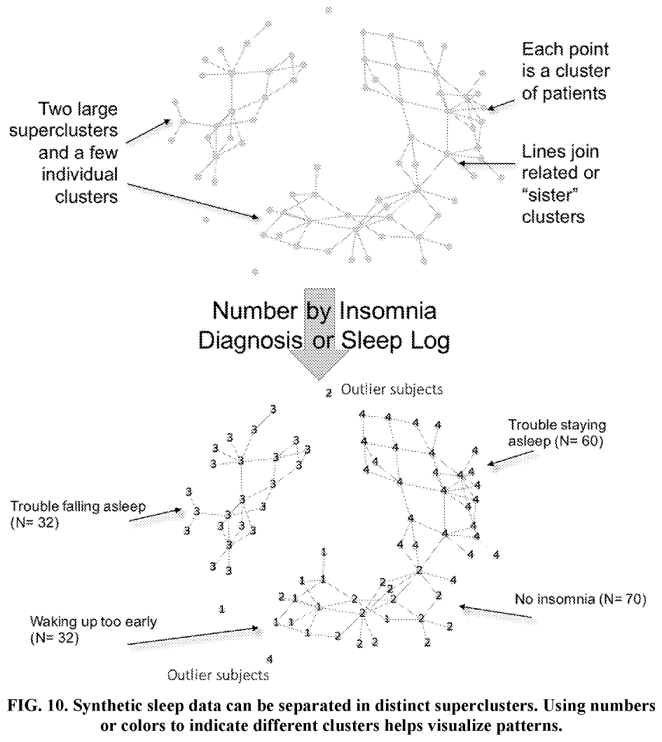

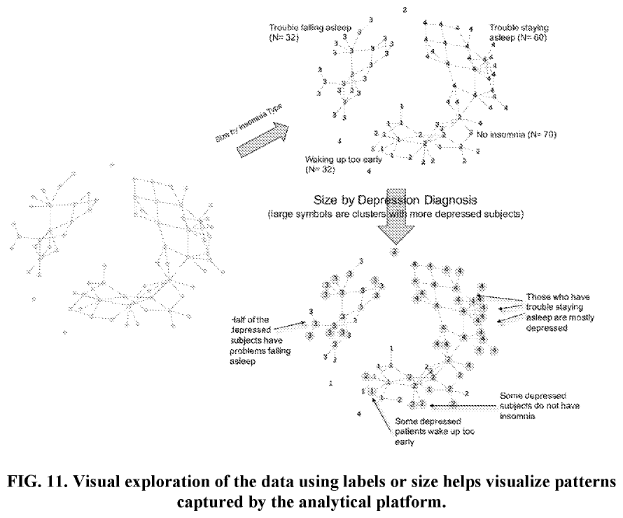

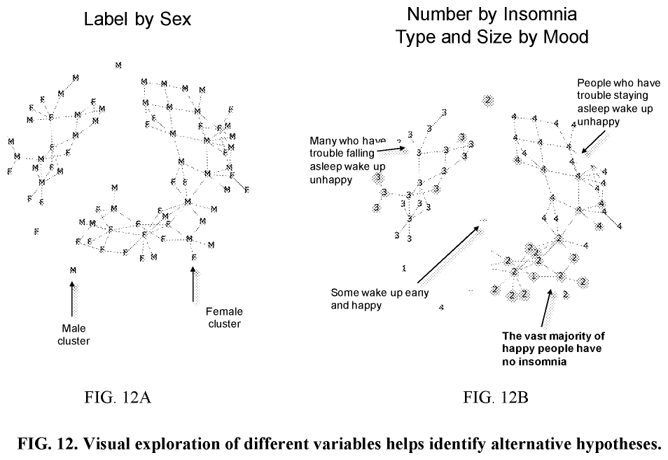

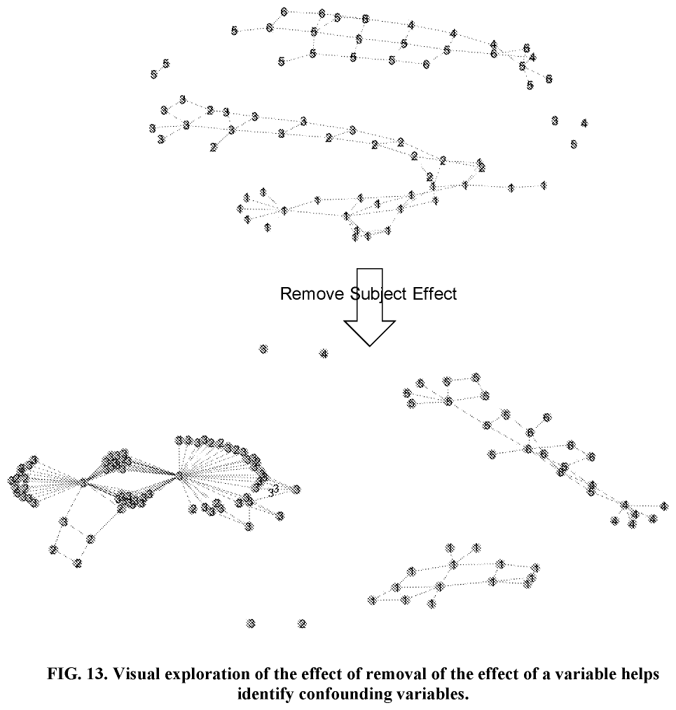

Since it is difficult to visualize objects of more than three dimensions, a dimension reduction process needs to take place, with the constraint of maintaining the local relationship between points (or patients) in the original point cloud. The latter is imperative for visual identification of trajectories, and deviation from them. The analytical platform can satisfy these needs using dimensionality reduction if needed (using for example principal component analysis or clustering methods such as ENCLUS) and appropriate visualization tools such as multidimensional scaling, Reeb Graphs, Contour Trees, topological data analysis [Ref. 19].Topological Methods for the Analysis of High Dimensional Data Sets and 3D Object Recognition) or the like. The visual outcome, for example, can be a network in which individual patient, or group of patients, are clustered into nodes, with edges connecting nodes with overlapping patient's populations. The general structure of the network together with the localization pattern of the patients across the network can be used to define and identify those patterns and classes, and provide hints on the underlying disease mechanisms. FIG. 8 exemplifies a network depicting different insomnia types (see Data Analysis Example 1), visualized with TDA after PCA dimensionality reduction, in which clusters are composed of subjects presenting with similar sleep patterns. For example, using labels, patterns, symbols, color, size, or other markers according to a known diagnosis or label (e.g. “depressed” versus “control”; FIG. 11) allows for exploration of the interpretation of the visual output. As can be seen in FIGS. 10-12, such visual patterns can then be quantified to assess significance level of various parameters. The ability to visually present patterns, explore possible interpretations, and quantify pattern significance is a major advantage of the present invention. FIGS. 10-12 display various clusters created from the data shown in Example 9. In FIGS. 10-12, each point or “node” represents a cluster of patients. As can be seen the data can be segregated into common “related” or “sister” clusters which share one or more common features. Accumulation of multiple clusters may allow the formation of superclusters. In FIG. 10, two large super clusters are formed. Further imposing graphical information on these superclusters demonstrates the segregation between the three types of insomnia (here, each node is labeled with a 1, 2, 3, or 4 representing the three types of insomnia and control). FIG. 11 further qualifies the clusters allowing size to be proportional to the number of depressed subjects in each cluster, allowing a visualization of the depression x insomnia interaction. FIG. 12 explores the relationship with sex or mood. FIG. 13 shows the ability of the platform to classify different activities where now clusters represent datasets corresponding to one of 7 possible activities (1: Laying, 2: Sitting, 3: Standing, 4: Walking downstairs, 5: Walking upstairs, 6: Walking) and the improved performance obtained when a possible confounding variable (Subject) is removed from the model (erasing all dependencies between the rest of the variables in the model and the target confounding variable). With the removal of the Subject variable most inter-subject variability is accounted for and the separation of the different activities is improved. Bias removal allows assessing the effect of different variables on a putative classification, and also results in a more orderly dataset available for further analysis.

The user. The present invention has at least four types of users that may present with different queries, require different algorithms, and need different answers and visual representations. The subject: This person has interest in using the analytical platform to assess his/her own health status and trajectory. The results will be available on a smartphone, tablet, laptop, or similar devices, or submitted in writing. Subjects may have access to the raw and processed data and may be presented with comparisons between his/her health status and a baseline or population data. FDA-approved recommendations can also be included. In addition, the analytical platform can automatically and programmatically trigger, or be complemented with, electronic access to particular therapy of proven efficacy (such as a CBTI APP provided for PTSD patients). The caregiver: Similarly, a caregiver (relative, nurse, counselor, or the like) may want to have access to a particular analysis, report, or visualization. The health care provider: A doctor or health system manager may have very different needs in terms of analysis. For example, special prospective (e.g., prognosis) and retrospective analyses (e.g. research on early predictors of a heart attack) can be provided for these users. The researcher: a researcher may want to explore dependencies between variables of no obvious value to the other users, in order to better understand the disorder, improve further data collection, optimize therapy development, explore complementary analyses, or generate hypotheses. For example, visually exploring the data may reveal that subjects presenting with a particular disease have a heart rate variability that is not correlated with activity, and that, in turn, may suggest a particular physiological deficit, which may be then amenable to experimental research. Access to the analytical platform, its tools and visualization output, can therefore be customized to fit the needs of each user. The present invention incorporates all such needs considering both streaming (data analysis in, or almost, real time) and static analyses of data (delayed analysis), a flexible toolbox of algorithms, and varied visual representations. The core platform serves all users.

Bias reduction. The art of data analysis includes the important process of bias detection and identification of confounding variables. Bias may be the consequence of lack of control of an environmental variable, such as temperature, or subject variable, such as sex. For example, activity data patterns may be strongly influenced by environmental temperature. Ignoring temperature may lead to the erroneous conclusion that, for example, diagnosis of depression does not correlate with changes in sleep architecture. It is possible that if temperature is included in the model underlying the data analysis, such correlation may appear or be strengthened.

Alternatively, data can be transformed to remove all dependencies with temperature and the analysis can be focused just on the variable of interest. This second approach is particularly appealing when the confounding variable is of no interest in itself such as bias introduced by differences in experimental protocols, measurement instruments, or clinical study site. Methods for bias removal include the simplest z-score, to remove differences in the central value and variance of two or more data distributions. Regression techniques can also be used to remove a trend due to a variable of no interest. FIG. 13 (Data example 2) shows the increased differentiation between activity categories after bias removal using PCA and TDA to visualize the data.

Tables IV and V specify examples of data utilized by the system and the various stages of processing data.

TABLE IV
Types of data referred to in this invention

| | | | | |
|---|---|---|---|---|
| Data | Ex- | Objective | Continuous, | Heart data, EEG, EKG, |
| | perimental | | Passive | EMG, galvanic skin |
| | | | | response, electrolytes, |
| | | | | analytes, acceleration, |
| | | | | activity, etc |
| | | | Discrete | Memory test, taping test, |
| | | | | etc |

| | | |
|---|---|---|
| | Subjective | Emotion, confidence, |
| | | mood, well-being, |
| Metadata | Contextual | Medication, education, |
| | | diagnosis, prognosis, |
| | | disease status, disease |
| | | progression, place of |
| | | residence, coordinates, |
| | | time of day, etc |
| | Environmental | Temperature, humidity, |
| | | weather, etc |
| | Subject | Gender, age, race, name |
| | Descriptive | Study number, study |
| | | title, experimental |
| | | details, keywords |
| | Structural | Number of data records, |
| | | number and identification |
| | | of data records subsets |
| | Administrative | Upload and download date, |
| | | database origins, file |
| | | type, data format, |

TABLE V
Stages of data processing

| | | | | |
|---|---|---|---|---|
| Data | Non-ag- | Raw | Binary | Data as captured by the sensor |
| | gregated | | | without any processing |
| | | | Clean | Binary data with basic processing |
| | | | | such as band filtering, artefact |
| | | | | removal, etc |

| | |
|---|---|
| Processed | Data processed to identify |
| | particular events or states, their |
| | quantification, timing, frequency, |
| | count, etc |

| | |
|---|---|
| Ag- | Data summarized over a short on long |
| gregated | period, means, variability, etc |
| Derived | Data inferred from non-aggregated or |
| | aggregated data such as correlations, |
| | imputations, extrapolations, etc |

While several embodiments of the invention have been discussed, it will be appreciated by those skilled in the art that various modifications and variations of the present invention are possible. Such modifications do not depart from the spirit and scope of the claimed invention.

This specification incorporates by reference herein all publications, patents and patent applications mentioned herein, to the same extent if the specification had specifically and individually incorporated by reference each such individual publication, patent or patent application.

While several embodiments of the invention have been discussed, it will be appreciated by those skilled in the art that various modifications and variations of the present invention are possible. Such modifications do not depart from the spirit and scope of the claimed invention.

### EXAMPLES

### Imputation Example 1. Using Multivariate Higher Order Moments

The difficulty that missing values present is that imputed values can bias the dataset in unknown ways. For example, replacing missing values with simple variable means (first order moment) is likely to reduce the variable variance (second order moment), which may differentially affect the goodness of fit of different models. It is of interest therefore to preserve higher order moments of the individual variables (e.g. variance, skewness and kurtosis) as well as the relationship of different variables.

The simplest second order moment for two variables x, y, is the covariance between the two variables and their respective variances. This is captured in a 2×2 covariance matrix CV

$$C V = ( \begin{matrix}C V ( x , x ) & C V ( x , y ) \\ C V ( y , x ) & C V ( y , y )\end{matrix} )$$

where CV(x,y) is the expected value of (x−<x>)*(y−<y>), the covariance of x and y; CV(x,x) is the expected value of (x−<x>)*(x−<x>), the variance of x; and CV(y,y) is the expected value of (y−<y>)*(y−<y>), the variance of y.

In general, when there are n variables, the second order moment is captured by an N×N covariance matrix (CV). If higher order moments are desired to be captured, one could also calculate the co-skewness (CS) with an N×N×N matrix and co-kurtosis (CK) with an N×N×N×N matrix http://www.quantatrisk.com/2013/01/20/coskewness-and-cokurtosis/) of n variables.

In a preferred embodiment, an imputing algorithm will choose values, for example, by bootstrapping [Ref 20] that do not significantly change the observed estimates of the higher order moments, as well as the normally considered lower moments.

As an example, consider that three variables x, y, z with zero mean have a covariance matrix:

$$C V = ( \begin{matrix}1 & 0 & 0 \\ 0 & 1 & 0 \\ 0 & 0 & 1\end{matrix} )$$

meaning that the pairs (x,y), (y,z) and (x,z) do not covary (the CV is zero). It is entirely possible that even in this case the pair (x,y) show low values when z is low, and high when z is high. In other words, the pair (x,y) depends on the value of the third variable. Thus, in this case:

CV(x,y)=CV(x,z)=CV(y,z)=0 and CS(x,y,z)>0.

The point of the example is to show that a simple matrix of covariances does not contain all the information contained in the n-dimensional space of the n variables considered, and that higher order moments of single and multivariables can and may be considered for improved imputation.

A test of the amount of bias added by the technique can be performed by attempting to classify the data with and without imputation. That a classifier of choice performs significantly better when classifying labeled data, or above chance when classifying unlabeled data can be used as indication that imputation introduced bias and a different method needs to be used.

Alternative methods to capture higher order relationships comprise mutual information [Ref. 21], partial correlation [Refs. 22], and conditional expectation [Ref 23].

### Imputation Example 2. Using Interpolation and Regression Models

Another way to estimate values that will improve model fitting without introduction of bias is to consider each variable trajectory. Trajectory for each variable can be estimated using simple, multiple or fractional polynomial regression models [Ref 24]. Using the latter, for example, it is possible to fit a nonlinear function to a variable (such as heart rate as a function of day in the year) using covariates to produce a better estimate (such as time of day, gender, body weight, etc.). Once the optimal model is found, missing values can be estimated by interpolation or extrapolation.

### Analysis Example 1. Personal Trajectories and Deviation from Expected Value

One of the preferred embodiments comprises the analysis of a longitudinal personal dataset with health-related information collected over a period of days, months or years. The subject in this example may be a healthy person who decides to use a wearable device to track his health. Using the device, he connects to the analytical platform described in this invention and starts recording and getting feedback on his data. During the first few days there is not enough information to build a personalized model; however, the data can be compared against a database of data belonging to a healthy normal population and to other databases that represent different disorders. Using trained classifiers an early assessment can be made of his data and the feedback may consist of his classification as a healthy person or a probability that the person has a certain disease. In this case, however, the preferred use is to track a patient's own trajectory, which can be modeled after a minimal period of use of the wearable device. The personal trajectory is not build on a single parameter but on a combination of all his data. This integrated profile can be defined using simple, multiple or fractional polynomial regression models, for example [Ref. 25]. Using these or other methods an estimate of the expected trajectory can be drawn by extrapolation of the model parameters. Such prediction can be then compared with newly obtained data, as the subject continues to use the wearable device, to obtain a prognosis. For example, prediction based on current data may indicate a stable health trajectory, yet data obtained after analysis was first performed shows deterioration of the overall personal profile prompting for further analysis to extract, if possible, specific domains that explain the sudden change, and/or a visit to the doctor for further data gathering, or treatment. In this example, the analytical platform sends not only feedback about an unexpected change but also points to body weight as being the driver of the abnormal change. The subject can therefore bring this weight issue to his doctor and provide extensive data and analysis from the analytical platform, showing that body weight has changed, although other domains also captured by this particular wearable device have not. The doctor may order follow up exams that may, for example, show gastrointestinal inflammation, and may prescribe and antibiotic or other treatment and a change in diet.

### Analysis Example 2. Personal Trajectories and Deviation from Norm

Another preferred embodiment comprises the analysis of a longitudinal personal dataset but a comparison of the expected personal trajectory against the normal (or specific disease) population trajectory (FIG. 6, middle panel). Such comparison can be done once the dataset for the population is sufficiently large to estimate population parameters. As an example, consider a woman who is diagnosed with a certain type of cancer. After successful treatment, the doctor suggests continuous monitoring of vital signs using a couple of particular wearable gadget invented in the doctor's hospital. She then starts using the devices, logs her data into the analytical platform, and starts monitoring her profile on a daily basis. As an example of the imputation step, consider, for example, that she loses one of the devices and thus, loses a week of data until she obtains a new one from her doctor and continues monitoring all requested data. Using the imputation methods described in this invention the missing data is modelled and added to her dataset for analysis of her trajectory. In this example, a comparison of her personal data versus the population health trajectory may indicate a normal profile for several months, giving the subject peace of mind. However, after several months her profile starts to change and deviates from the healthy population. This automatically triggers further analysis (although the subject can request in depth analysis at any time) to extract the specific domains that explain such deviation from normal, and comparison of her deviant profile against the various disease databases existing in the system. The profile may now resemble more that of a cancer population rather than the healthy population. This immediately triggers a visit to the doctor who orders new clinical analyses, which may reveal recurrence of the cancer, and lead to the start of a new treatment round.

### Analysis Example 3. Personal Trajectories and Abrupt Changes

Yet another preferred embodiment comprises the analysis of a longitudinal personal dataset and extraction of temporal change points for which the system specifies a change larger than expected (FIG. 6, top panel). A person, such as the woman and man in the two examples above, may monitor his or her health trajectory using the system described in this invention. A general health deterioration (detected as a change from the stable trajectory) may be found through the analysis of the dataset as a whole, and could be later tracked down to a specific change in a particular domain. For example, a deviation of the personal trajectory from the predicted or from the normal may not be gradual but abrupt, and the in-depth analysis may point to the cardiovascular data as the earliest variable to change abruptly (such as it would result from the onset of cardiac arrhythmia), leading in the short term to deterioration of other domains (e.g. activity, sleep, EEG). Cardiovascular data acts in this example as a leading indicator of an upcoming general deterioration. The Ensemble Metalearner Module 17 may place more weight on algorithms with particular sensitivity to such shifts such as change point detection [Ref. 26], or Likelihood Ratio algorithm [Ref. 27]. It is also possible to fit the data with simple, multiple or fractional polynomial regression models within a time window, and redo the fit with a shifted time window (i.e., a moving window). The model parameters can then be analyzed to detect an abrupt change. In this case, as the system analyses individual data with the best trajectory algorithms and finds deviations from the norm or the predicted individual trajectory, it may send an automated query that triggers the in-depth analysis leading to the use of change point algorithms for detection of the earliest significant deviations, and the identification of the leading indicators. All these results can then be sent to the user or attending clinician via a user interface.

Change points can be continuous transitions or discontinuous transitions (called bifurcations when they involve two distinct states), and different models may provide differential sensitivity, so the system provides a variety of readily applicable algorithms. Defining what type of transition has been found may give insight into the type of process driving the change in trajectory. For instance, it is possible that in a certain disease heart rate is either cyclic or has a particular type of arrhythmia, with no value in between, constituting a two-state system. These cyclic patterns can be summarized using topological data analysis, or other suitable modeling techniques, and enter the system as secondary data.

It is important to extract information regarding the leading indicators, as this information could be crucial to further the understanding the causes of the general deterioration, as well as to quantify the thresholds that determine a significant change in the trajectory. Leading indicators can be found exploring the contribution to the model fitness given by the different domain data, contextual and/or other data. For example, analysis may indicate a change in ambient temperature occurred shortly before the change point, and that, despite variability in other variables, temperature is the best statistical predictor of the change point. It is possible also, for example, that only the cardiovascular domain is found to contribute to the general profile change point (all other domains being stable), suggesting a more circumscribed health problem. Quantitative analysis can find that when ambient temperature crosses 80° F., for example, then certain type of individuals experience considerable worsening of their symptoms. As it can be seen, a change point finding can trigger a series of secondary analyses that provide important insight into change point interpretation.

### Analysis Example 4. Personal Trajectories Between Normal and Disease Population Trajectories

Yet another example can be given in which a treatment needs to be assessed in, for example, a clinical trial. A person may be given a treatment for a disease condition and it is therefore of personal and medical interest to consider the individual trajectory with respect to both the disease population and the normal population trajectory (FIG. 6. Bottom panel). The personal trajectory can be analyzed against the disease population baseline looking for change points indicating a departure from the expected disease trajectory (beneficial or side effect effects). A comparison against the normal population trajectory adds to the interpretation of such change, with movements towards the norm being indicative of a beneficial treatment effect. Further analysis of the change point may confirm that the treatment onset is the leading indicator, and no other possible changes (such as a change in ambient factors). Such information would be of great value for the clinical trial director, as now the factors affecting the individual trajectories of subjects recruited into the clinical trial can be added to the analysis to explain more of the variance in the data, leading to more statistically robust results. For example, in a clinical trial for depression, it is possible that a novel antidepressant treatment lead to normalization of the sleep cycle. As this happens during the period the subjects are not being observed as part of the clinical trial, such information would be lost unless the participants use an activity or EEG device that tracks circadian signals or EEG associated with sleep. In our example, all participants are asked to wear such devices, and thus the beneficial normalization of the sleep cycle is captured. It may be the case that measurements of activity and mood, done with the wearable devices or even in the clinical setting, show a beneficial effect of treatment on activity patterns and mood. As it is known in this example that the normalization in sleep occurred before other changes, it is possible for the scientists involved in the clinical trial to speculate and further investigate the hypothesis that the mechanism of action of the antidepressant in first directed towards sleep mechanisms, and only secondarily to mood. Such ability to extract continuous information, trajectory deviations, change points, and leading indicators would revolutionize clinical trials. In particular, it will lead to the reduction of the placebo effect, as it would be impossible for participants to deviate from their own depression trajectory, in this example, all the time. That is, there will be point in the day or the week in which depressed participants assigned to the placebo group will show their true depressed state, whereas those assigned to the treatment group will show the beneficial effects of treatment.

### Analysis Example 5. Group Comparisons

Personal trajectories are not the only analyses of interest. It should be clear that group analyses are also of great interest and that the system described in this invention is amenable to such investigations. These include the comparison between two or more different groups, such as, but are not limited to, a normal versus a disease group, a young versus an old group, a male versus a female group. The questions being asked to the system could be, but are not limited to, “which are the most important domains that separates two groups under consideration”, “what is the time course of the data belonging to such most important domains”, “is there a change point in the disease trajectory that defines critical disease periods to be considered for treatment onset”, and/or, “is a particular treatment being more efficacious than another”.

### Analysis Example 6. Cross-Sectional Comparisons

It should be also clear that trajectories are of particular interest due to the power to predict the future embedded in the longitudinal datasets but that point cross-sectional analyses can also be performed. These include, but are not limited to, a comparison between a subject and a normal population at a given age, comparison of two groups at the end of a treatment, and other such point analysis.

### Data Example 1

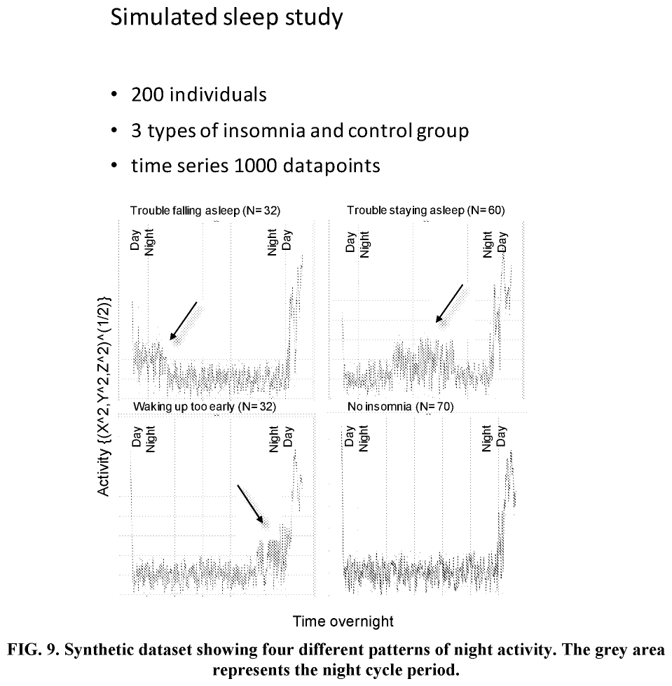

To showcase the ability of the platform to quickly identify patterns in time series data we analyzed a synthetic dataset composed of 200 samples of x, y, z coordinates simulating a 3-axes accelerometer. Random uniform noise (range 0-1) was added throughout the 200 time series samples, but three out of four subsets had random noise (range 0-2) added at the beginning of the series, middle, or end, to simulate bouts of insomnia at the beginning, middle, or end of the sleep cycle, with the forth subset serving as control with no insomnia. In addition, higher levels of deescalating and escalating activity were randomly programmed at the beginning and end of the synthetic night period (FIG. 9). Data was analyzed in the platform using PCA dimensionality reduction and TDA for visualization. The resulting cluster network for the insomnia data illustrates two superclusters: one that groups clusters of subjects that wake up too early (“1”), have trouble staying asleep (“4”), or having a normal sleep pattern (“2”, FIG. 10). The second supercluster uniformly shows all subjects that had trouble falling asleep (“3”). As can be seen insomnia due to people having trouble falling asleep primarily forms its own supercluster. A second variable (e.g. depression diagnosis) and its interaction with the first (in this case, sleep pattern) can be explored, e.g., setting cluster symbol size to be proportional to the percent of depressed people in such cluster (FIG. 11). Imposing multiple data on each cluster using size, color or other markers allows further correlation to be drawn. In FIG. 12, insomnia data on the left is demonstrated with the nodes labeled as male (“M”) or female (“F”). The right figure further illustrates the correlation between subject's mood and insomnia, with the size of the node representing the average mood for each cluster. Such illustration allows various previously unidentified interactions to be drawn between disparate variables. Thus, it is possible to visualize that depressed subjects have trouble staying asleep (FIG. 11), that people who sleep well are happier (FIG. 12A) or that insomnia and sex are unrelated (FIG. 12B) e.g., correlations that can then be analyzed, quantified, and explored experimentally.

### Data Example 2

To showcase the ability of the platform to remove the effect of unwanted, biasing, or confounding variables, a dataset consisting of 3-axis accelerometer and 3-axis gyroscope time series data, and corresponding parameters resulting from a Fourier transform [Ref. 28] was processed and visualized. FIG. 13 shows that the platform can separate clusters corresponding to different gestures, and that application of an algorithm removing the effect of the variability between subjects greatly improved the separation between gesture classes.

### REFERENCES CITED AND ALTERNATIVE EMBODIMENTS

References listed below are those indicated throughout the text with the “[Ref]” notation.
1. A brief history of wearable computing (http://www.media.mit.edu/wearables/lizzy/timeline.html#1268); Georgia Tech. “Smart T-shirt”, Nov. 14, 1997, Georgia Institute of Technology Press Release (http://www.gtwm.gatech.edu/gtwm.html); Hawley, Michael, R. Dunbar Poor, and Manish Tuteja. “Things that think.” Personal Technologies 1.1 (1997): 13-20.
2. Personal Health Monitor for Homes, April 1997, Timo Tuomisto & Vesa Pentikainen, ERCIM News, No. 29. (http://www.ercim.eu/publication/Ercim_News/enw29/tuomisto.html)
3. Newman-Toker, David E., and Peter J. Pronovost. “Diagnostic errors—the next frontier for patient safety.” JAMA 301.10 (2009): 1060-1062.
4. King, Gary, et al “Analyzing incomplete political science data: An alternative algorithm for multiple imputation.” American Political Science Association. Vol. 95. No. 01 Cambridge University Press, 2001; Schafer, Joseph L., and John W. Graham. Missing data: our view of the state of the art. Psychological methods 7.2 (2002): 147.
5. Kotsiantis, Sotiris B., I. Zaharakis, and P. Pintelas. “Supervised machine learning: A review of classification techniques.” (2007): 3-24; Kononenko, Igor. “Machine learning for medical diagnosis: history, state of the art and perspective.” Artifcial Intelligence in Medicine 23.1 (2001): 89-109; Scheffer, M., Carpenter, S. R., Lenton, T. M., Bascompte, J., Brock, W., Dakos, V., et al. (2012). Anticipating Critical Transitions. Science, 338, 344-348.
6. Parisi F1, Strino F, Nadler B, Kluger Y. Ranking and combining multiple predictors without labeled data. Proc. Natl. Acad. Sci. USA. 2014 Jan. 28; 111(4):1253-8. DOI: 10.1073/pnas.1219097111. Epub 2014 Jan. 13; Tumer, Kagan, and Joydeep Ghosh. Error correlation and error reduction in ensemble classifiers. Connection science 83-4 (1996): 385-404; Whalen, Sean, and G. K. Pandey. A comparative analysis of ensemble classifiers: case studies in genomics. DataMining (KCDM), 2013 IEEE 73th International Conference on. IEEE, 2013.
7. Wearable body monitor device with a flexible section and sensor therein” USPTO Application #20140275813; A brief history of wearable computing (http://www.media.mit.edu/wearables/lizzy/timeline.html#1268); Georgia Tech. “Smart T-shirt”, Nov. 14, 1997, Georgia Institute of Technology Press Release (http://www.gtwm.gatech.edu/gtwm.html)
8. Grundy, Betty L., et al. “Telemedicine in critical care: an experiment in health care delivery.” Journal of the American College of Emergency Physicians 6.10 (1977): 439-444; and Hawley, Michael, R. Dunbar Poor, and Manish Tuteja. “Things that think.” Personal Technologies 1.1 (1997): 13-20; Personal Health Monitor for Homes, April 1997, Timo Tuomisto & Vesa Pentikainen, ERCIMNews, No. 29. (http://www.ercim.eu/publication/Ercim_News/enw29/tuomisto.html)
9. Autosense. https://sites.google.com/site/autosenseproject/
10. Mobilize Center. http://mobilize.stanford.edu/
11. James Walker, Fourier Analysis and Wavelet Analysis, Notices of the AMS, V 44, N6
12. http://www.quantatrisk.com/2013/01/20/coskewness-and-cokurtosis
13. Lonardi, Jessica Lin Earnonn Keogh Stefano, and Pranav Patel. “Finding motifs in time series.” Proc. of the 2nd Torkshop on Temporal Data Mining. 2002.
14. Xu, Rui, and Donald Wunsch. Survey of clustering algorithms. Neural Networks, IEEE Transactions on 16.3 (2005): 645-678.
15. Troyanskaya, Olga G., et al. A Bayesian framework for combining heterogeneous data sources for gene function prediction (in Saccharomyces cerevisiae). Proceedings of the National Academy of Sciences 100.14 (2003): 8348-8353.
16 Paulsen, Jane S. “Cognitive impairment in Huntington disease: diagnosis and treatment.” Current neurology and neuroscience reports 11.5 (2011): 474-483.
17. Possin, Katherine L. “Visual spatial cognition in neurodegenerative disease.” Neurocase 16.6 (2010): 466-487.
18. F. Carrillo, G. Bedi, G. A. Cecchi, D. F. Slezak, M. Sigman, N. Mota, S. Ribeiro, D. C. Javitt, M. Copelli and C. Corcoran “Automated Analysis of Free Speech Predicts Psychosis Onset in High-Risk Youths”, NPJ Schizophrenia, 2015; F. Carrillo, N. Mota, M. Copelli, S. Ribeiro, M. Sigman, G. A. Cecchi, D. Fernandez Slezak, “NIPS—Machine Learning and Interpretation in Neuro Imaging” (2014), Lecture Notes in Artificial Intelligence—Springer; Bedi G, Cecchi G A, Fernandez Slezak D, Carrillo F, Sigman M, de Wit H, “A Window into the Intoxicated Mind?Speech as an Index of Psychoactive Drug Effects”, Neuropsychopharmacology, 2014; N.B. Mota, N. A. P. Vasconcelos, N. Lemos, A. C. Pieretti, O. Kinouchi, G. A. Cecchi, M. Copelli, S. Ribeiro, “Speech Graphs Provide a Quantitative Measure of Thought Disorder in Psychosis”, PLoS One, 2012.
19. Gurjeet Singh, Facundo Mémoli, & Gunnar Carlsson. Eurographics Symposium on Point-Based Graphics (2007) M. Botsch, R. Pajarola
20. https://en.wikipedia.org/wiki/Bootstrapping_(statistics))
21. “Estimation of mutual information using kernel density estimators,” YI Moon, B Rajagopalan, U Lall—Physical Review E, 1995—civil.colorado.edu
22. Partial correlation estimation by joint sparse regression models; J Peng, P Wang, N Zhou, J Zhu—Journal of the American Statistical Association; Volume 104, Issue 486, 2009)
23. Evolution without evolution: Dynamics described by stationary observables, DN Page, WK Wootters—Physical Review D, 1983—APS; Models for longitudinal data: a generalized estimating equation approach; SL Zeger, KY Liang, PS Albert—Biometrics, 1988—JSTOR
24. Regression Using Fractional Polynonials of Continuous Covariates. Parsimonious Parametric Modelling Patrick Royston and Douglas G. Altman. Journal of the Royal Statistical Society. Series C (Applied Statistics) Vol. 43, No. 3 (1994), pp. 429-467 Published by: Wiley for the Royal Statistical Society DOI: 10.2307/2986270 Stable URL: http://Nvww.jstor.org/stable/2986270
25. Regression Using Fractional Polynomials of Continuous Covariates: Parsimonious Parametric Modelling Patrick Royston and Douglas G. Altman Journal of the Royal Statistical Society. Series C (Applied Statistics) Vol. 43, No. 3 (1994), pp. 429-467 Published by: Wiley for the Royal Statistical Society DOI: 10.2307/2986270
26. Choi & Chukkapalli, Applying Machine Learning Methods for Times Series Forecasting; Proceedings of the IASTED International Conference Artificial Intelligence and Applications, 2009
27. Computation and analysis of multiple structural change models, Bai & Perron, Journal of Applied Econometrics, 2003
28. https://archive.ics.uci.edu/ml/datasets/Smartphone+Dataset+for+Human+Activity+Recognition+(HAR)+in+Ambient+Assisted+Living+(AAL)

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that comprises a computer program mechanism embedded in a nontransitory computer readable storage medium. Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1-33. (canceled)

34. A method for monitoring a brain health status of a subject, the method comprising:
at a computer system comprising one or more processors and a memory:
receiving, in electronic form, information on a baseline health of the subject through, at least in part, results of a clinical assessment;
obtaining, in electronic form, a first plurality of observables of the subject taken across a period of time, wherein the period of time comprises a plurality of time intervals;
obtaining, in electronic form, a second plurality of observables of the subject across the period of time, wherein the first plurality of observables and the second plurality of observables are different from each other and are each selected from the group consisting of (i) a plurality of sensor measurements obtained from a sensor associated with the subject, (ii) a plurality of measurements of amounts of one or more analytes in the subject, (iii) a plurality of physiological measurements of the subject, and (iv) a plurality of subjective observations by the subject;
for each respective time interval in the plurality of time intervals, using at one least observable in the first plurality of observables and at least one observable in the second plurality of observables taken during the respective interval to compute a respective brain health index value set for the subject, wherein the respective brain health index value set comprises two or more of (i) a motor skill, (ii) a cognitive skill, (iii) a mood, and a (iv) sleep status; and
determining one or more brain health trajectories of the subject across the period of time through a comparison of the respective brain health index value set at each time interval in the plurality of time intervals to the baseline health of the subject.

35. The method of claim 34, wherein the determining the one or more brain health trajectories of the subject further comprises refining the respective one or more brain health trajectories based on a comparison to a plurality of reference brain health index value sets, wherein each respective reference brain health index value set in the plurality of reference brain health index values set represents a reference subject in a plurality of references subjects that is calculated using observables of two or more of i) a sensor measurement obtained from a sensor worn by the respective reference subject, (ii) a measurement of one or more analytes of the respective reference subject, (iii) a physiological measurement of the respective reference subject, and (iv) a subjective observation by the reference subject.

36. The method of claim 34, wherein the respective brain health index value set for the subject is computed as output from one or more models upon inputting the at one least observable in the first plurality of observables and at least one observable in the second plurality of observables into the one or more models.

37. The method of claim 34, wherein the information on the baseline health of the subject further comprises a neuroimage of the brain of the subject obtained by a neuroimaging technique.

38. The method of claim 37, wherein the neuroimaging technique is electroencephalography.

39. The method of claim 34, wherein the clinical assessment of the subject indicates that the subject has depression.

40. The method of claim 34, wherein the clinical assessment of the subject indicates that the subject brain trauma.

41. The method of claim 34, wherein the clinical assessment of the subject indicates that the subject has anxiety.

42. The method of claim 34, wherein the clinical assessment of the subject indicates the subject has post-traumatic stress disorder.

43. The method of claim 34, wherein the clinical assessment of the subject indicates the subject has Alzheimer's Disease.

44. The method of claim 34, wherein the first plurality of observables or the second plurality of observables is a plurality of sensor measurements obtained from a sensor worn by the subject and measures a galvanic skin response of the subject.

45. The method of claim 34, wherein the first plurality of observables or the second plurality of observables is a plurality of measurements of amounts of one or more analytes in the subject.

46. The method of claim 34, wherein the first plurality of observables or the second plurality of observables is a plurality of physiological measurements of the subject and each physiological measurement in the plurality of physiological measurements is a heart rate of the subject.

47. The method of claim 34, wherein the first plurality of observables or the second plurality of observables is obtained from a sensor associated with the subject.

48. The method of claim 47, wherein the sensor is
a wrist band with a wireless transmitter worn by the subject,
a physiological sensor attached to the subject,
an injectable sensor that is injected into the subject,
an ingestible sensor that is ingested by the subject,
a shoe sensor worn by the subject,
an eye tracking device in visual communication with the eyes of the subject,
a smart-shirt worn by the subject, or
a computerized textile worn by the subject.

49. The method of claim 47, wherein the first plurality of observables or the second plurality of observables obtained from the sensor associated with the subject measures movement of the subject, dexterity of the subject, or speech of the subject.

50. The method of claim 34, wherein the first plurality of observables or the second plurality of observables is recorded by a smart phone associated with the subject.

51. The method of claim 34, wherein the period of time is one minute or greater, five minutes or greater, one hour or greater, one day or greater, or one week or greater.

52. A computer system comprising one or more processing cores and a memory, the memory storing instructions for performing a method for monitoring a brain health status of a subject, the method comprising:
receiving, in electronic form, information on a baseline health of the subject through, at least in part, results of a clinical assessment;
obtaining, in electronic form, a first plurality of observables of the subject taken across a period of time, wherein the period of time comprises a plurality of time intervals;
obtaining, in electronic form, a second plurality of observables of the subject across the period of time, wherein the first plurality of observables and the second plurality of observables are different from each other and are each selected from the group consisting of (i) a plurality of sensor measurements obtained from a sensor associated with the subject, (ii) a plurality of measurements of amounts of one or more analytes in the subject, (iii) a plurality of physiological measurements of the subject, and (iv) a plurality of subjective observations by the subject;
for each respective time interval in the plurality of time intervals, using at one least observable in the first plurality of observables and at least one observable in the second plurality of observables taken during the respective interval to compute a respective brain health index value set for the subject, wherein the respective brain health index value set comprises two or more of (i) a motor skill, (ii) a cognitive skill, (iii) a mood, and a (iv) sleep status; and
determining one or more brain health trajectories of the subject across the period of time through a comparison of the respective brain health index value set at each time interval in the plurality of time intervals to the baseline health of the subject.

53. A non-transitory computer-readable medium storing one or more computer programs executable by a computer for monitoring a brain health status of a subject, the one or more computer programs collectively encoding computer executable instructions for performing a method comprising:
receiving, in electronic form, information on a baseline health of the subject through, at least in part, results of a clinical assessment;
obtaining, in electronic form, a first plurality of observables of the subject taken across a period of time, wherein the period of time comprises a plurality of time intervals;
obtaining, in electronic form, a second plurality of observables of the subject across the period of time, wherein the first plurality of observables and the second plurality of observables are different from each other and are each selected from the group consisting of (i) a plurality of sensor measurements obtained from a sensor associated with the subject, (ii) a plurality of measurements of amounts of one or more analytes in the subject, (iii) a plurality of physiological measurements of the subject, and (iv) a plurality of subjective observations by the subject;
for each respective time interval in the plurality of time intervals, using at one least observable in the first plurality of observables and at least one observable in the second plurality of observables taken during the respective interval to compute a respective brain health index value set for the subject, wherein the respective brain health index value set comprises two or more of (i) a motor skill, (ii) a cognitive skill, (iii) a mood, and a (iv) sleep status; and
determining one or more brain health trajectories of the subject across the period of time through a comparison of the respective brain health index value set at each time interval in the plurality of time intervals to the baseline health of the subject.
